SEQUENCE LISTING (73 sequences)

SEQ ID NO: 1 - DNA, 2007 bp, Arabidopsis thaliana
  CDS (1)..(2007)
atgcagatttgccaaaccaagctcaatttcactttccctaatcccacaaaccctaatttctgcaaacccaaagctcttcaatggtcaccgcctcgtcgcatatccttgctgccttgtcgtggattcagctccgatgaattcccagtcgacgaaaccttcctcgagaaattcggaccaaaggacaaagacacagaagatgaagctcgacgacgtaactggatcgaacgtggttgggctccatgggaagagattctcacaccagaagctgatttcgctcgtaaatctctcaacgaaggtgaagaagttccgcttcaatcgccggaagcgatcgaagcgtttaagatgctgagaccatcgtataggaagaagaagattaaggagatggggataacagaagacgaatggtatgcaaagcaatttgagattagaggtgataaaccacctcctttagaaacatcttgggctggtccgatggttcttaggcaaattccgccgcgtgattggcctcccagaggttgggaagttgataggaaggagctggagtttattagggaagctcataagttaatggctgaaagagtttggcttgaggatttggataaggatttgagagttggtgaagatgctactgttgataagatgtgtttggagaggtttaaggttttcttgaaacaatacaaggaatgggttgaagataataaagataggttggaggaagaatcttacaagctcgatcaggatttttatccgggtaggaggaaaagagggaaggattacgaagatgggatgtatgagcttcccttttactatccagggatggtttgtgaaggcacagttaccactttacatctgtatcagggagcgtttgttgacattggaggtgttcatgaaggatgggtacctataaaaggtaatgactggttttggatccggcatttcataaaagttgggatgcatgttatcgttgaaatcacggcaaaaagagatccataccggtttcggtttcccttggagttgcgcttcgtccatcctaacatagatcacatgatatttaataaatttgacttcccaccaatattccatcgtgatggggatactaatccagatgagatacggcgagattgtggaagacctcctgaacctagaaaagatccaggatcaaagccagaggaggaagggctgctctctgatcacccttatgtcgacaagttgtggcagatacatgtagctgagcaaatgattttgggtgattacgaagctaaccctgcaaaatacgaaggcaaaaagctatcagaattatctgatgatgaagactttgatgaacaaaaggatatcgagtatggcgaagcttattataagaaaaccaaattgccaaaagtgattctgaaaaccagtgtcaaggaacttgacttagaggctgcattgaccgagcgccagcaccacaataaactaatgatggaagctaaagcaagaggagaaggatacaaaattgacaagctcagacgaaatatagagatggacgagtatgatttcttacactggcgccgatctttggaggaaagagaagcattgctcagagatatcagctctcggcaagcacttggtttaccattggaggaaccagggaggtacaagccaggaagcttctttgggaaagaccagtacgatccaacaagtgcattatatcagtatgactactggggagagccaaagaactcagagattagcaagcaagagagaatgaaggatgcacacaacaaatccattgttgggaaaggcaatgtgtggtatgacatgtcttacgatgatgcgattaagcagacaatagagaaaagaaaagaagggtctaccttggcgagtcaagaagaagaaacagagtcagaggaagaagaagaggatgacgatgattttgacgattttgactatagcattctgagtgatgagagcagtatcggttactcggaacagcaacctcttgttaacggtactcaagtcttgacagactga

SEQ ID NO: 2 - protein, 668 aa, Arabidopsis thaliana
MQICQTKLNFTFPNPTNPNFCKPKALQWSPPRRISLLPCRGFSSDEFPVDETFLEKFGPKDKDTEDEARRRNWIERGWAPWEEILTPEADFARKSLNEGEEVPLQSPEAIEAFKMLRPSYRKKKIKEMGITEDEWYAKQFEIRGDKPPPLETSWAGPMVLRQIPPRDWPPRGWEVDRKELEFIREAHKLMAERVWLEDLDKDLRVGEDATVDKMCLERFKVFLKQYKEWVEDNKDRLEEESYKLDQDFYPGRRKRGKDYEDGMYELPFYYPGMVCEGTVTTLHLYQGAFVDIGGVHEGWVPIKGNDWFWIRHFIKVGMHVIVEITAKRDPYRFRFPLELRFVHPNIDHMIFNKFDFPPIFHRDGDTNPDEIRRDCGRPPEPRKDPGSKPEEEGLLSDHPYVDKLWQIHVAEQMILGDYEANPAKYEGKKLSELSDDEDFDEQKDIEYGEAYYKKTKLPKVILKTSVKELDLEAALTERQHHNKLMMEAKARGEGYKIDKLRRNIEMDEYDFLHWRRSLEEREALLRDISSRQALGLPLEEPGRYKPGSFFGKDQYDPTSALYQYDYWGEPKNSEISKQERMKDAHNKSIVGKGNVWYDMSYDDAIKQTIEKRKEGSTLASQEEETESEEEEEDDDDFDDFDYSILSDESSIGYSEQQPLVNGTQVLTD

SEQ ID NO: 3 - DNA, 1161 bp, Arabidopsis thaliana
  CDS (1)..(1161)
atgagtaagagacctccgcctgatccagtggctgtgcttagagggcaccgtcactctgttatggatgtttccttccatccatcaaaatctctgcttttcactggttcagctgatggagagttgaggatttgggatactattcagcaccgtgcagtttcttctgcttgggctcatagtagagcaaatggggttttagctgtagcagctagtccctggttaggagaggacaagattatcagccagggaagagatggaactgttaagtgttgggatattgaagatggtggtctatcaagagacccattactaatacttgagacatgcgcctatcatttttgcaagttctctcttgttaagaagcctaagaattctctgcaagaagctgaaagtcactcacggggttgtgatgagcaggatggtggagacacatgtaatgttcaaattgctgatgatagtgaacgatctgaggaggattctggtttgcttcaggataaagatcatgctgaaggtaccacttttgtagcagtagtaggggagcaacctactgaggtagagatatgggatctcaataccggagacaagataatacagcttcctcaaagtagccctgacgaatcccccaatgcttctaccaaaggaagaggaatgtgtatggctgttcaattgttttgccctcctgaatcacagggtttcttacatgtcttagctggttatgaggatggttcaatacttctttgggatatacgcaatgctaagattcctctaacaagtgtgaagtttcattcagagccagttttaagtcttagtgttgcatcatcatgtgatggaggaatctcaggaggggcagatgacaaaattgtgatgtataatctcaaccattcaactggttcttgtacaataaggaaagagattactttggaaaggcccggtgtatcgggcacctcaattcgagtcgatgggaaaattgcagccacggctggttgggaccacagaatacgagtctataattaccgcaaaggaaatgctctagcaatactaaagtaccaccgagcaacgtgcaacgctgtgtcctactctccagactgtgagctaatggcgtctgcgtctgaagatgcaactgttgctctctggaaactatatcctccccataaatctctctga

SEQ ID NO: 4 - protein, 386 aa, Arabidopsis thaliana
MSKRPPPDPVAVLRGHRHSVMDVSFHPSKSLLFTGSADGELRIWDTIQHRAVSSAWAHSRANGVLAVAASPWLGEDKIISQGRDGTVKCWDIEDGGLSRDPLLILETCAYHFCKFSLVKKPKNSLQEAESHSRGCDEQDGGDTCNVQIADDSERSEEDSGLLQDKDHAEGTTFVAVVGEQPTEVEIWDLNTGDKIIQLPQSSPDESPNASTKGRGMCMAVQLFCPPESQGFLHVLAGYEDGSILLWDIRNAKIPLTSVKFHSEPVLSLSVASSCDGGISGGADDKIVMYNLNHSTGSCTIRKEITLERPGVSGTSIRVDGKIAATAGWDHRIRVYNYRKGNALAILKYHRATCNAVSYSPDCELMASASEDATVALWKLYPPHKSL

SEQ ID NO: 5 - DNA, 2700 bp, Arabidopsis thaliana
  CDS (1)..(2700)
atggtgtccctctctaatcaaaccagattctctttccacccaaacaatcttgtcgtttccgagaaacggcgcctgggaatctcaggagtcaattttcccaggaagatcaagttgaaaatcacttgcttcgccgccgagagaccacgccaggagaaacagaaaaagaagagtcagagccagagcactagcgacgctgaggctggagttgaccctgttgggtttctcaccagactcggaattgctgacagaatctttgctcaatttctccgggaaaggcataaagctttgaaagaccttaaagatgagattttcaaacgccatttcgatttcagagattttgcttcaggatttgagttattggggatgcatcgacatatggaacaccgggttgactttatggactggggtccaggttctcgttatggggctataatcggagatttcaatggatggtcgcctacagaaaatgcagctagagagggcctatttggccatgatgattatgggtattggttcattattcttgaagataagttaagagagggtgaagaaccagatgaactatactttcagcagtataactatgtcgatgactatgataagggtgatagtggtgtatcagctgaagaaatcttccagaaagctaatgatgagtattgggaacctggtgaggatcggtttattaaaaaccgctttgaagttcctgccaaactatacgagcagatgtttggcccgaacagtccacaaacgttagaagagcttggagatataccagatgcagaaacgagatataaacagtggaaagaggagcataaagatgacccgccaagtaatctacctccatgcgacatcattgataaaggtcaaggaaagccatatgatatttttaatgtcgtgacatctccagaatggacaaagaaattttatgagaaagaacctccgattccatattggttagagacacgtaaaggaagaaaggcatggttgcaaaaatatattccagctgttccacatggaagcaagtatagattgtatttcaatactcctgatggaccgcttgaacgagtgcctgcttgggctacatatgtgcaaccagaggatgaaggaaagcaagcttacgcaattcattgggaaccttctcctgaagctgcatacaagtggaaatattccaagccaaaagttccagagtccttgcgcatatacgaatgtcatgttggaatcagtgggtccgagccaaaagtttcaactttcgaagaatttactaagaaggtccttcctcatgtgaaaagagctggatacaatgcaatccagttgattggtgtccctgagcacaaggattattttactgttggttatagggttacgaacttctttgctgccagtagccgatatggcacgccagatgatttcaaacggttggttgatgaggcacatggcctaggacttcttgtctttttggacatcgtgcattcttatgcagcagctgatcagatggttgggctttctctcttcgatggttcaaatgattgctattttcattatggtaaaagggggcatcacaaacactggggcacccggatgttcaaatacggtgatttggatgttcttcattttctaatatcaaatttgaactggtggattacagagtatcaagttgatggttaccaatttcactcgcttgcctcgatgatttacacgcacaatgggtttgcttcatttaacaacgatttggatgactattgcaatcagtatgttgaccgagatgctctgatgtacctcattttggccaatgaaatcctgcacgttcaacatccaaatataataacaattgctgaggatgcaacatattaccctgggttgtgtgagccagtttctcaaggtggacttggatttgattattatgtgaatctttctgcgtcagaaatgtgggtttctctcctcgacaatgtaccggataatgaatggagcatgagcaagattgtcagtacattggtggctaacaaagagtatgcagacaagatgctcagctatgccgaaaatcacaaccaatccatatcaggagggcgttcatttgctgaaatcttattcggtggagtcgataatggttctcctggaggaaaagaattgctagacaggggaatttcactacataagatgattagactgattacttttacaagtggtggccgtgcatacctcaatttcatgggaaatgaatttggacatcctgagagggttgagtttccaacgcagagcaataacttctcgttttcacttgctaaccgccgctgggacctgctggaaagtggagtgcatcatcacttgttttcctttgacaaggagctaatggacttggataaaagcaagggtatcctttcaagaggtctgcccagcatccaccatgtgaatgatgcaaatatggtgatttctttctcaaggggtcctttcctatttatctttaacttccacccatcaaattcatatgaaaagtatgatgtcggtgtagaggaagctggtgaatataccatgatactgaactccgatgaagtaaagtatgggggtcaagggattgtaacagaagaccactatcttcaacggtcaattagcaagagaattgatggtcaaagaaattgcttagaggtgtttttgcctagccggactgcccaagtttacaagttgacccggatcctccgaatatga

SEQ ID NO: 6 - protein, 899 aa, Arabidopsis thaliana [sequence omitted]

SEQ ID NO: 7 - DNA, 903 bp, Arabidopsis thaliana
  CDS (1)..(903)
atggtgctacttctccgatatcgatccttaacgataaatctcactcctctaatacccaaatctcaaaaattccacactctccaatcatttcgtaaccctaatttcatctccatcccaaaaatctcagcctccacgaacaatccaacaacaacaacgaatcggtcgatctccgacgcgactaaattcgctaagtcagtgttattcatacctcctggtgtagagattgaggaactaacagatgatatggttcttccgggttcaaatatcgtaatcggaccttttgcgggtcattcacaaatcaaagaagttgaatttgttaaaagtagtgctcgtgctagagattgtcctaaagatgatcgtcctgagattgcgattttgggtcgttctaatgttggcaagtcttcgcttatcaattgtttggttcgtaagaaagaagttgctcttacttctaagaaacctgggaagactcagcttataaatcacttcttggtgaataagagttggtacattgtggatttgcctggttatggatttgctaaagtttcagatgctgcgaaaacagattggtctgcgtttactaaaggttacttcttgaatagagacagtcttgtttgtgttctacttctcatcgatgcaagtgttcctcctcagaagattgatcttgattgtgctaattggcttggtcgcaacaatgtaccgatgactttcgtgtttacgaaatgcgataaaatgaaagcaacgaaagggaaaagacctgatgagaacattaaagctttccaacaaatcattagagagaattttaaagtacatcctccttggattttgacgagtagtgtatctggtttaggcagagacgagcttcttcttcacatgtcgcagctgagaaactactgggatcaatag

SEQ ID NO: 8 - protein, 300 aa, Arabidopsis thaliana
MVLLLRYRSLTINLTPLIPKSQKFHTLQSFRNPNFISIPKISASTNNPTTTTNRSISDATKFAKSVLFIPPGVEIEELTDDMVLPGSNIVIGPFAGHSQIKEVEFVKSSARARDCPKDDRPEIAILGRSNVGKSSLINCLVRKKEVALTSKKPGKTQLINHFLVNKSWYIVDLPGYGFAKVSDAAKTDWSAFTKGYFLNRDSLVCVLLLIDASVPPQKIDLDCANWLGRNNVPMTFVFTKCDKMKATKGKRPDENIKAFQQIIRENFKVHPPWILTSSVSGLGRDELLLHMSQLRNYWDQ

SEQ ID NO: 9 - DNA, 2412 bp, Arabidopsis thaliana
  CDS (1)..(2412)
atgatgtcggtgagagcaatcaatggttgctccatcattcgcaccgccacttccgccggcggtcctcccgtctctctattccgccaccgtattcagcgcctcagagcttctcatctccgtgaattctcgaaattacgtttaaattttccactaatccgcgcggataggcggtttctcggaaatagtgatgctccgagctgttcgacttgcatacatagtctggtcgaaagtgtttccgaagagctcgaatccatcagcagacgcaagggaagccgaatgagagttcgcgccagtgtaaaggtgaaattaacaagctacggagaggttctagaggataagctagtaaaccaggagctagaggctggtttgcttctggagtttaagaaggacgcagacagagtgttgttggcagttcttcatagacgtgatggcaagaagaattggatggtgtttgatcagaatggtgttagttgttccatcaaaccacagcaaattacatacatcgtcccgaatgtttataattttgatcatacaggacttactgattttctccaaagggctcaagataacttggatccccaactgcttgagtttgcttggatggagttacttgagaagaacaaacctgtgaccccagaagagttagcagagatgatatatggtcgcgcagatcctcttgaaagctattgtgctcattttttgctatcacaagacgagatatacttttctatcttggagtcaaaaggttctcgatcaatatatagccctcggcctactgaacaggtggaagagcttttacgaaggcaacgcgtgaaggaggcagaagataaagagtttcaggagtttattcaattgctgaagtctgccaagaaggcacctagtcacgccaagccccctaaatcttcatggctagctgatgataaagtccaagataggattggctcacttgaagcttatgccattgatgcatgggcaagcactgaccagcagaaattggctggaacgatattaaaatccatggggttacagaaaacgtctgtatcagcactaaaccttctcatagatattggatattttcctgtacatgtcaatcttgaactgcttaagctgaatcttccaacccaccactcagaagctatcactgaagctgctgaagctcttctttcagaatcgtctgatattgatgcggttaggaggattgatctcacacacttgaaggtttatgcaattgatgttgatgaggctgatgagcttgatgatgccctcagtgcaacgagattgcaggatggacggataaagatctggatacatgttgctgatcctgctagatatgttacacctgggagtaaagtggacagagaggcaaggagaaggggaacttctgtctttctgccaacggctacttatccaatgtttccagagaaacttgccatggaaggaatgagcttaagacaaggagaaaactgtaatgctgtctctgtgtctgttgtcctgcgttctgatggctgtattacagaatattcagtagataattcaatcatcagaccgacctatatgttgacgtatgaaagtgcttctgagctgcttcacttgaacctagaagaagaagctgaactgaaattgctgtctgaggcagcattcattcgctctcagtggcgccgtgaacagggtgcagtggacacaactacactagaaactcgtatcaaagtggtgaatcctgaggatccagaacctttgataaatctctatgtagaaaatcaggcagatctagcgatgagacttgtctttgaaatgatgattctttgtggggaggttgtagcaacgtttgggtctcagcataatattccattgccatatagaggacagccacagtcaaatattgatgtatctgcatttgcgcatcttccagaaggaccagtacgttcctcatccattgtcaaagtaatgcgtgctgcagaaatgaatttcagatgtcctgttcgacatggagtgcttggaattcctggttacgttcagttcacgtctcccattcgtcgatacatggatcttactgcacactatcagattaaagcttttcttcgaggaggtgacaattttccattctctgctggtgagttagaaggtatagcagcatccgtaaacatgcagagcaaagtggtgagaaaactctcaaacaccggtctccgctattgggtaatagagtttctcagaagacaggaaaagggtaaaaagtacaccgcattggtcttgagatttgtcaaagaccggatcgcatcccttttgttggttgaggtgggttttcaagcaacggcgtgggtgtcagaaggaaaacaagtgggagatgaaatcgaagttcgagtagaagaagctcatccccgcgacgatcttattctcttcaaagaggttatctag

SEQ ID NO: 10 - protein, 803 aa, Arabidopsis thaliana
MMSVRAINGCSIIRTATSAGGPPVSLFRHRIQRLRASHLREFSKLRLNFPLIRADRRFLGNSDAPSCSTCIHSLVESVSEELESISRRKGSRMRVRASVKVKLTSYGEVLEDKLVNQELEAGLLLEFKKDADRVLLAVLHRRDGKKNWMVFDQNGVSCSIKPQQITYIVPNVYNFDHTGLTDFLQRAQDNLDPQLLEFAWMELLEKNKPVTPEELAEMIYGRADPLESYCAHFLLSQDEIYFSILESKGSRSIYSPRPTEQVEELLRRQRVKEAEDKEFQEFIQLLKSAKKAPSHAKPPKSSWLADDKVQDRIGSLEAYAIDAWASTDQQKLAGTILKSMGLQKTSVSALNLLIDIGYFPVHVNLELLKLNLPTHHSEAITEAAEALLSESSDIDAVRRIDLTHLKVYAIDVDEADELDDALSATRLQDGRIKIWIHVADPARYVTPGSKVDREARRRGTSVFLPTATYPMFPEKLAMEGMSLRQGENCNAVSVSVVLRSDGCITEYSVDNSIIRPTYMLTYESASELLHLNLEEEAELKLLSEAAFIRSQWRREQGAVDTTTLETRIKVVNPEDPEPLINLYVENQADLAMRLVFEMMILCGEVVATFGSQHNIPLPYRGQPQSNIDVSAFAHLPEGPVRSSSIVKVMRAAEMNFRCPVRHGVLGIPGYVQFTSPIRRYMDLTAHYQIKAFLRGGDNFPFSAGELEGIAASVNMQSKVVRKLSNTGLRYWVIEFLRRQEKGKKYTALVLRFVKDRIASLLLVEVGFQATAWVSEGKQVGDEIEVRVEEAHPRDDLILFKEVI

SEQ ID NO: 11 - DNA, 3888 bp, Arabidopsis thaliana
  CDS (1)..(3888)
atgacgcagtcgcctgttcaaccgcctccgccgctcccagctcagccgcattccgccgccggaggtgtaataagaggcgatgtacaggttaggtgcgcaggctgtcgcgtgattctgagggtgaaaaccggcgttgtggagttctcgtgtccgacttgtcaacttccccaaatgttgccgccggagcttctttctagggcacggccgcagtttccgcagtctccacaacaaccaccacagccaattcagacgcttccgccaccgattcaacagcagcttaagcctcttaatctacctaggcctccggttccggcacatggaatcgatccgacgaagatgcaattgccttgcgctaactgtcaggctatcctcaatgtgcctcatggactcactcggttctcgtgtcctcagtgccatgtcgagctcgctgttgatgtctccaagcttaatcggtctttaactgcttctcaatctcattctaatcctcctactcctgctgctcccacagtcccgcctccgcctcctcccgaagaagtcaatgaggaagctattgaagtggagagagaagaagatgaaggtggaacggctggagaaacgtttatggattatcgtcccccaaaactttcgatcggacctcctcatcctgatccaattgtggaaacatcatctctatctgcagtgcaacccccagagcctacttatgatctcaaaatcaaggaagagcttgaaaggtcaaaggctttatcgtgtttgcagattgagacattagtttacgcttgccagaggcatctccagcaccttgctgatggtaccagagcaggcttttttgttggagatggtgctggtgtgggcaaagggcgaacaattgccgggttgatttgggaaaattggaaacatggaaggaggaaagctttgtggatttctattggctcagacttgaaatatgatgcaagaagggacttggatgatgtgggtgctacatgcgttggagtgaatcctttgaacaagctaccctattctaagcttgactcgaaaaatgttggtattaaagaaggagtggtattcttgacatacaatagcctcattgcttcctctgagaagggccgttctcgtttacagcagcttgttcaatggtgtggaccagaatttgatggtctcttgatctttgatgagtgccataaagcgaaaaatttggtacctgaagctgggagtcagccgacacgcatcggacaggcagttgttgacattcaggataagatcccccaagcacgtgttatttattgctcggctactggggcatcagaaccacgaaacatgggttatatggtcaggcttggtctttggggagctggaacaagttttagtgattttaacaaattcctaggtgcccttgataaaggtgggacaggagcgttggaattggttgccatggatatgaaagcgagggggatgtatgtatgccgaaccctgagttacaaaggggctgagtttgagattgttgaagctcgtttagaagcaggaatggaggcaatgtacaataagtctgcagaattttgggcagagctacggatagagttgttgtcagcaagtgcttttctccctaacgagaaaccaaattctagtcagttgtggagattatactggtcaagccaccagcgtttctttagacacctgtgtatgtcagctaaagtccctgtaactgtgaggttagctaagaaagccttgtcaacaaacaagtgtgtagttattggccttcagagtactggagaggctcgaaccgaagaggctgtaaataaatatggtcttgagcttgatgattttgtctcgggacctcgagagctcttgttgaaatttgtggaagagaattatcccttacccgagcagcctgaacctctgtcagaggatgatagtgttaaagaacttcagaggaagaggcattcagcttcacctggtgtttcaattagagggagagtaagaaagatggcaaagtggaaaccagatagtgataatgaaagtgatttggagtctgaagctgactctgctgacgattctaatgattctgatgatgagttccagatatgtcagatatgcagtggggaagatgaaaggaagaagttgctccattgttctgaatgtgacaagctttttcatccggactgtgtggttcctccagttatagacttaccatctgaagcgtggatttgcttttcttgtaaggaaaagacagaggagtacattcaagcaagacgtctttacattgccgaattgcagaaaaggtatgaagcagcgcttgaacgaaaatcaaagatcatagagatcatccgttctctgaatcttccaaacaatcctttggatgacatcgtcgatcagcttggaggccctgaaaaagttgcagaaatgacgggcaggcgaggtatgcttgtaagagcatcgaatgggaaaggtgttacatatcaagcaagaaacacaaaggatattaccatggaaatggtcaacatgcacgagaagcaactatttatggatggtaaaaagttggttgccattatttctgaagctgggtcagctggtgtttcgttgcaagcagatagaagggcagtaaatcagaaaagaagggttcacttgactttagagcttccctggagtgctgatcgtgcaattcagcaatttggaagaactcaccggtcaaatcagacatctgcacctgaatatagactactcttcactaatctcggtggggaacgccgctttgcctctattgttgcaaagagactagaaacacttggtgctttaacacagggagaccgcagggctgggccctccgggccttctttgagtgcgtataattatgatagtaactttggtaaaaaatccttgatggtgatgtatcgaggcataatggagcaggagaaactacctgttcttcctcctggatgctcgattgatgaacccgaaacagttaaagagtttttgaccaaggcaagggctgctcttgttgcggttggaattgttagggacagtgttctggccaatgggaaagatgttggaaggttttcaggacgtattattgattctgacatgcatgatgttggacgatttttgaatcgcctcttaggattgccacctgatatccagaataggctgtttgaattgtttactagtatattagacgttcttgtacataatgctcggatagaaggaagttttgactcaggaattgtggatatgaaagctaatagtgttgagctactgagcactccaaagaccgtccatgttgatcaaatgtctggtgcctcaactatgctctttacttttactctagatcgtggtgtaacatgggagtctgcgagttcaatgctagagggaaagagaagggacgggcttggttcagctaatgatggcttttttgaatctaaaagggaatggttaggaagacggcacttcatattagcatttgagagtgccgcttctgggttgtttaagattgtacgtcctgctgtaggagagtcgatcagggagatgtctctttcagagttgaaaactaaataccgaaaactatcttctctggagaaggctcgtactggttgggaggacgagtacgaagtttcatcaaaacagtgtatgcatggacccaagtgtaagcttggggagtattgcacggttggacgtaggatacaggaagtaaacgttgtgggtggccttattcttcccatctggggaaccatcgagaaagctctctccaaacaagcccgtcacagccacaagagaattcgagttatacgcatagagactacgacggataatcagcggatagtgggcctctccatcccaaatgcagctgtagaaactgttttacaagatttagcatgggttcaagagattgatgattaa

SEQ ID NO: 12 - protein, 1295 aa, Arabidopsis thaliana
MTQSPVQPPPPLPAQPHSAAGGVIRGDVQVRCAGCRVILRVKTGVVEFSCPTCQLPQMLPPELLSRARPQFPQSPQQPPQPIQTLPPPIQQQLKPLNLPRPPVPAHGIDPTKMQLPCANCQAILNVPHGLTRFSCPQCHVELAVDVSKLNRSLTASQSHSNPPTPAAPTVPPPPPPEEVNEEAIEVEREEDEGGTAGETFMDYRPPKLSIGPPHPDPIVETSSLSAVQPPEPTYDLKIKEELERSKALSCLQIETLVYACQRHLQHLADGTRAGFFVGDGAGVGKGRTIAGLIWENWKHGRRKALWISIGSDLKYDARRDLDDVGATCVGVNPLNKLPYSKLDSKNVGIKEGVVFLTYNSLIASSEKGRSRLQQLVQWCGPEFDGLLIFDECHKAKNLVPEAGSQPTRIGQAVVDIQDKIPQARVIYCSATGASEPRNMGYMVRLGLWGAGTSFSDFNKFLGALDKGGTGALELVAMDMKARGMYVCRTLSYKGAEFEIVEARLEAGMEAMYNKSAEFWAELRIELLSASAFLPNEKPNSSQLWRLYWSSHQRFFRHLCMSAKVPVTVRLAKKALSTNKCVVIGLQSTGEARTEEAVNKYGLELDDFVSGPRELLLKFVEENYPLPEQPEPLSEDDSVKELQRKRHSASPGVSIRGRVRKMAKWKPDSDNESDLESEADSADDSNDSDDEFQICQICSGEDERKKLLHCSECDKLFHPDCVVPPVIDLPSEAWICFSCKEKTEEYIQARRLYIAELQKRYEAALERKSKIIEIIRSLNLPNNPLDDIVDQLGGPEKVAEMTGRRGMLVRASNGKGVTYQARNTKDITMEMVNMHEKQLFMDGKKLVAIISEAGSAGVSLQADRRAVNQKRRVHLTLELPWSADRAIQQFGRTHRSNQTSAPEYRLLFTNLGGERRFASIVAKRLETLGALTQGDRRAGPSGPSLSAYNYDSNFGKKSLMVMYRGIMEQEKLPVLPPGCSIDEPETVKEFLTKARAALVAVGIVRDSVLANGKDVGRFSGRIIDSDMHDVGRFLNRLLGLPPDIQNRLFELFTSILDVLVHNARIEGSFDSGIVDMKANSVELLSTPKTVHVDQMSGASTMLFTFTLDRGVTWESASSMLEGKRRDGLGSANDGFFESKREWLGRRHFILAFESAASGLFKIVRPAVGESIREMSLSELKTKYRKLSSLEKARTGWEDEYEVSSKQCMHGPKCKLGEYCTVGRRIQEVNVVGGLILPIWGTIEKALSKQARHSHKRIRVIRIETTTDNQRIVGLSIPNAAVETVLQDLAWVQEIDD

SEQ ID NO: 13 - DNA, 1038 bp, Arabidopsis thaliana
  CDS (1)..(1038)
atggtgttggtaacccataaattgcagggttcacatgtcttgtctccttggagttcgccaacatggaccagaggcttgtgtttaaagagacctgttacgacagtgcactttgttgagaggaaagataagcatttacgattgaagcaaaaatgttgttttagtttagggtccccttgcaacggtggattgagagcaaaacctttcagagttacatcttttaaaggtggcatccaaaataatgaatcaggaggaagtgagggtgggaagaaagttactaataactctgtcaaactatcttaccgttcagatgatgacgaaaataatgtgaatggctccccaaaggcacagaacacttcgttttcatatacctcggaaacagaggattccatgacagggcaacccgctattcagaaactattcaagaaatggttgacattgttgcgcacacagtcacctattcaagtgattgacgagactttaggaggagaacaggttccacaaacgacgaagcaagaaactgaaacagaaattcggaaagcagaaagtctccagagtaccaaaaatacagtttggtcctggttttggagtctagacgcagctatcaagattccgttgctgttatttgttccagctttcctagctgtttacgcgatctgtggtgctgaagttacaaaggaattatcccctatgtgggtggttggccccttgatcatcgccctttatataaaaatgttccaaggactatgttccctttacgccttctgcttcaaccaaaccatcaaaatgataagaaacctaccatcgtattatcttgtagcataccaatacattgccgatggaaagctcaaagacgacgttaaagctctagtgtttaaaccagtggtggccatcaagaacacagactacaaagagctcacacgcacgaagctgaaacagttccaagaatggatcattgagaaatacctggattttgttgaatctgtatggccttattactgcagaactatcagattcctgaagagggctaacctgatttga

SEQ ID NO: 14 - protein, 345 aa, Arabidopsis thaliana
MVLVTHKLQGSHVLSPWSSPTWTRGLCLKRPVTTVHFVERKDKHLRLKQKCCFSLGSPCNGGLRAKPFRVTSFKGGIQNNESGGSEGGKKVTNNSVKLSYRSDDDENNVNGSPKAQNTSFSYTSETEDSMTGQPAIQKLFKKWLTLLRTQSPIQVIDETLGGEQVPQTTKQETETEIRKAESLQSTKNTVWSWFWSLDAAIKIPLLLFVPAFLAVYAICGAEVTKELSPMWVVGPLIIALYIKMFQGLCSLYAFCFNQTIKMIRNLPSYYLVAYQYIADGKLKDDVKALVFKPVVAIKNTDYKELTRTKLKQFQEWIIEKYLDFVESVWPYYCRTIRFLKRANLI

SEQ ID NO: 15 - DNA, 6474 bp, Arabidopsis thaliana
  CDS (1)..(6474)
atggaaggggatgagcgaggagtcttacttgcttgtgtaatttcgggtacccttttcacggttttcggttcgggttcgttttggatactttgggctgttaattggcggccatggcgtctctacagttggatctttgctagaaaatggccaaaagtattgcaaggtcctcagcttgatatactatgtggtgttctatctctttttgcttggattgtggtagtatcccctattgcaatcttgataggatggggttcttggctgattgtgatattggatcgacatatcattgggctggcgataataatggctggarcagcccttttactggcattctactcaatcatgctttggtggaggacccagtggcaaagctcaagagctgtcgctttacttctccttcttggtgttgccttactatgtgcgtatgaactctgtgctgtctatgttacggctggtgcgcatgcatctcagcaatattctccttctggtttctttttcggtgtatcagcaatcgcgttggcaattaacatgctatttatctgccgcatggtctttaatggaaatggtttagatgtggacgaatatgtaaggagggcatataaatttgcttattcagattgtatagaagtgggtcctgtggcttgtttgcctgaacctcctgatcctaatgaattatatccccggcaaaccagcagggcttcacatcttggccttctgtacctgggctcactcgtagttctccttgcctactcagtcctatatggtctcacagctagggaatcacgttggcttggaggaatcacatcagctgcagttattgttcttgactggaatattggggcatgcttgtatgggtttaagcttcttcagaatcgtgttctggcactttttgttgctggcatatcccgtcttttcctaatatgttttggcatacactactggtacctagggcattgtattagttacattttcgtagcatcagttctatcaggtgctgctgtttctcggcatctatctataacagacccatcagctgcaagaagagatgccttacagagcacagtgatccgcttgagagaaggttttcggagaaaagagcagaatagttcttcaagttcttcagatggttgtggctcaagtataaaaagaagtagtagtatcgatgctggccatactggttgtactaatgaagcaaatcgtacggcagaatcttgcacggctgacaatctaactcgaacaggcagctctcaggagggaatcaatagcgacaaaagcgaagaaagtggaagaccaagcttaggtttacgtagtagttcatgtcgttctgtggtccaagagcccgaagcaggaacgtcttattttatggacaaagtttctgatcaaaataacactcttgttgtttgttcgagcagtggtctagatagccaaggttacgagtctagcacatcgaattctgcaaaccagcagcttttggatatgaatttggctcttgctttccaggaccagttaaacaatcctaggatagcctcgatacttaagaagaaasmaaaagaaggtgatcttgaactgactaatttgctgcaagacaaggggttggaccctaactttgctgtaatgttgaaggaaaaaaacttggatcctggaaaaaacttgggtctnactatattggcactacttcagaggagtagtttggatgcagatagagatcaccgcgacaatactgatattacaatcattgactcaaacagtgttgacaatactttgccaaatcagatttctttatccgaagaattgagactccgtggactagagaagtggcttaagttgtctagacttcttctgcaccatgtagcggggacaccagagagagcatggggcctctttagtcttgtctttatccttgaaacaatcattgtggccatttttcgcccaaagaccatcacgattataaattctagtcatcaacagttcgaatttggtttctctgtgctgctattgtcacctgttgtctgttcaataatggcttttcttcggtctcttcaagttgaggaaatggccttgacatcaaaatctcgcaagtatggctttgttgcctggcttctgagcacatcagttggattgtcactctcgttcttgagtaaatcgtcagtacttctgggaatatccttgactgtgcccctcatggcagcatgcctgtctattgctgttcccatatggatgcataatgggtaccaattttgggttccacagttatcatgtggtgaccaggcaagagatttacgatctcccaggataaaggggtttattctttggatttgtgttgtgttgtttgcgggttctgtaatttctcttggtgcgattatatctgctaaacctttggatgatttaaagtataagctgtttagtgccagagaaaacaacgtcacgtcaccatatacatcttctgtataccttggttgggcaatgtcatctggaattgctttagtagttaccgccattctaccaatagtttcatggtttgcaacttataggttttcccactcttctgctgtctgtctcatgatattctcagttgttctcgtggcattttgtggaacttcatatttggaagttgtaaaatctagagatgatcagttgcccacaaagggtgatttccttgcggccttgcttccacttgcatgcattccggcgctgctttcactatgctgtgggatggttaaatggaaggacgattgttggatactctctcgaggtgtatatgttttcttttcaataggtcttcttcttctttttggtgcgatagcagctgtcattgcagtcaaaccatggacgataggcgtatcttttctcttagttcttttccttatggtggtaacaattggtgtaatccatctttgggcgtcaaacaatttctatttaaccaggaaacagacatcctttgtctgctttcttgctcttcttttgggtttggccgcattccttctcggatggcatcaagataaagcatttgctggagcatctgttggttactttacattcctgtctctgttggctggaagagcattagctgttcttctatccccaccaattgtagtatattctccaagggtgctaccagtatatgtctacgatgctcatgctgattgcggaaagaatgtcagtgctgcatttcttgtcctgtatggaattgctttggcaacagaaggctggggtgttgttgctagtctgataatttatcctccgtttgcgggtgctgctgtatcagctatcacccttgtagtagcctttgggtttgctgtttctcgcccatgtttgactcttgagatgatggaggttgctgtacgctttcttagcaaggatactatagtgcaagctatctctcgatctgccacgaaaacaagaaatgctctatccggcacgtattcagctccccaaaggtccgccagctctgcagctcttctggttggggatccctctgcaatgcgtgataaagcagggaactttgtgcttcctagagatgatgtcatgaaattaagggatcgtctcaggaacgaagaaagagttgctggatcaatcttctacaaaatgcaatgcaggaaaggattccgtcatgaaccacctacaaatgtagattatagaagagacatgtgtgcccatgcaagagttttggcactggaagaggcaattgatacagaatgggtgtatatgtgggacaaatttggtggttatttactactattgttaggtttgacagctaaggcggagagagttcaggatgaggtacggttgcggctcttcttagatagcattgggttctcggatttaagtgccagaaaaatcagtaaatggaagccagaggatagaagacaattcgaaattattcaagagagttatctgagagagaaagagatggaagaggaaagccttatgcagagacgtgaagaagaagggagaggtaaagaaagaaggaaagctcttttggagaaggaagagcgcaaatggaaggaaattgaagcgtcccttattccatctattcctaatgctggtagcagggaggcagcagccatggcagctgcaatacgtgctgttgggggtgattctgtccttgaggattccttcgcaagagagagggtctcgggtattgcacgtaggatacgcactgctcaactagaacgacgtgcacaacagactggaatatctggggcagtttgtgttcttgatgatgaaccaatgataagtggtaaacattgcggccaaatggactcaagtgtctgtcaaagtcagaagattagcttttccgttacagcaatgatccaatccgattctggacctgtatgtctttttggcactgaatttcaaaagaaagtatgttgggagattctggttgctggttctgagcaaggaattgaggctggccaagttgggcttaggttgataacaaaaggtgagaggcagacaaccgttgctagagagtggtatattggtgcaaccagcataactgatggaaggtggcatacagtgacaatcacaattgatgctgatgcgggggaagctacttgttacatagatggtgggtttgatggctaccagaatgggttacctctaagtattggcagtgccatttgggaacaaggagctgaagtttggttgggtgttaggccacctatagatgttgatgcattcgggagatcagatagtgatggcgtcgaatcaaagatgcatattatggatgttttcctttgggggaaatgcttaagtgaagaagaggccgcttctttgcatgcagccattggcatggctgacttagacatgattgatttgtctgatgacaattggcaatggacggattcaccccccagagtcgatggttgggatagtgatcctgccgatgttgatctctatgatagggatgacgtagattgggatggacaatattccagtgggaggaaaagaagatcaggtcgggattttgtaatgagtgtcgattcctttgccaggagacacaggaaacccaggatggagacacaagaagatataaatcaaagaatgcgttcagttgagttggctgtcaaagaagctctctctgcacgaggtgataagcaatttactgaccaggaatttcctccaaatgatcgctctttatttgtggatacacaaaatcccccatcaaaattgcaggttgtttctgaatggatgagacctgactccattgtgaaagaaaacggtagtgattcccgtccctgcctgttctctggggatgcaaatccttcagatgtttgccaggggcgtttgggggattgttggttcttaagcgccgttgcagttttgacagaggtttcacgaatatctgaagtgatcattactcctgaatacaacgaggaagggatctacactgttcgtttttgtattcagggtgagtgggttcctgttgttatcgatgactggattccatgtgaatcacctggtaaaccagcttttgctactagcagaaagctcaatgaactctgggtctccatggtggagaaagcatatgccaagctccatggttcttatgaggcactggaggggggactggttcaggatgctcttgtcgacctaactggaggagctggtgaggagattgacttgcggagtgctcaagcacaaatagatcttgcaagtggcagattgtggtctcaattgttacgttttaaacaagaggggttcttacttggtgctggaagtccatcaggatctgatgttcatgtatcttccagtggcattgtgcaagggcatgcttactccgtcttacaggtgagagaggttgatgggcacagacttgttcagattcgaaatccatgggctaatgaagttgagtggaatggtccctggtcagactcatccccagagtggactgataggatgaagcacaagctgaagcatgttccacagtcaaaagaaggtatattctggatgtcttggcaagatttccagattcatttcagatcaatatatgtgtgtcgggtttacccccgtgagatgcgctactctgtaaatggccaatggcgaggttatagtgccggtggctgccaagattatagctcatggcatcaaaatccacaattcaggctgagggcaactggttctgatgcatctttaccaattcatgtgttcatcaccttaactcagggcgtaggtttctcgagaacaactcctggatttcgtaactaccaatcaagccatgattcacagttgttctatatcggattgaggattcttaaaactcgtggacgtcgtgctgcttacaacatatttcttcatgaatctgttggtggaacagactatgtgaattcccgtgagatttcatgtgaaatggttcttgaccctgatcctaagggttatactattgtcccaaccacgatacacccaggggaagaagcaccttttgtcctttcagtcttcacaaaagcatccattgttcttgaagctttgtag

SEQ ID NO: 16 - protein, 2157 aa, Arabidopsis thaliana
  misc_feature (105)..(105): The ′Xaa′ at location 105 stands for Ala, or Thr.
MEGDERGVLLACVISGTLFTVFGSGSFWILWAVNWRPWRLYSWIFARKWPKVLQGPQLDILCGVLSLFAWIVVVSPIAILIGWGSWLIVILDRHIIGLAIIMAGXALLLAFYSIMLWWRTQWQSSRAVALLLLLGVALLCAYELCAVYVTAGAHASQQYSPSGFFFGVSAIALAINMLFICRMVFNGNGLDVDEYVRRAYKFAYSDCIEVGPVACLPEPPDPNELYPRQTSRASHLGLLYLGSLVVLLAYSVLYGLTARESRWLGGITSAAVIVLDWNIGACLYGFKLLQNRVLALFVAGISRLFLICFGIHYWYLGHCISYIFVASVLSGAAVSRHLSITDPSAARRDALQSTVIRLREGFRRKEQNSSSSSSDGCGSSIKRSSSIDAGHTGCTNEANRTAESCTADNLTRTGSSQEGINSDKSEESGRPSLGLRSSSCRSVVQEPEAGTSYFMDKVSDQNNTLVVCSSSGLDSQGYESSTSNSANQQLLDMNLALAFQDQLNNPRIASILKKKXKEGDLELTNLLQDKGLDPNFAVMLKEKNLDPGKNLGXTILALLQRSSLDADRDHRDNTDITIIDSNSVDNTLPNQISLSEELRLRGLEKWLKLSRLLLHHVAGTPERAWGLFSLVFILETIIVAIFRPKTITIINSSHQQFEFGFSVLLLSPVVCSIMAFLRSLQVEEMALTSKSRKYGFVAWLLSTSVGLSLSFLSKSSVLLGISLTVPLMAACLSIAVPIWMHNGYQFWVPQLSCGDQARDLRSPRIKGFILWICVVLFAGSVISLGAIISAKPLDDLKYKLFSARENNVTSPYTSSVYLGWAMSSGIALVVTAILPIVSWFATYRFSHSSAVCLMIFSVVLVAFCGTSYLEVVKSRDDQLPTKGDFLAALLPLACIPALLSLCCGMVKWKDDCWILSRGVYVFFSIGLLLLFGAIAAVIAVKPWTIGVSFLLVLFLMVVTIGVIHLWASNNFYLTRKQTSFVCFLALLLGLAAFLLGWHQDKAFAGASVGYFTFLSLLAGRALAVLLSPPIVVYSPRVLPVYVYDAHADCGKNVSAAFLVLYGIALATEGWGVVASLIIYPPFAGAAVSAITLVVAFGFAVSRPCLTLEMMEVAVRFLSKDTIVQAISRSATKTRNALSGTYSAPQRSASSAALLVGDPSAMRDKAGNFVLPRDDVMKLRDRLRNEERVAGSIFYKMQCRKGFRHEPPTNVDYRRDMCAHARVLALEEAIDTEWVYMWDKFGGYLLLLLGLTAKAERVQDEVRLRLFLDSIGFSDLSARKISKWKPEDRRQFEIIQESYLREKEMEEESLMQRREEEGRGKERRKALLEKEERKWKEIEASLIPSIPNAGSREAAAMAAAIRAVGGDSVLEDSFARERVSGIARRIRTAQLERRAQQTGISGAVCVLDDEPMISGKHCGQMDSSVCQSQKISFSVTAMIQSDSGPVCLFGTEFQKKVCWEILVAGSEQGIEAGQVGLRLITKGERQTTVAREWYIGATSITDGRWHTVTITIDADAGEATCYIDGGFDGYQNGLPLSIGSAIWEQGAEVWLGVRPPIDVDAFGRSDSDGVESKMHIMDVFLWGKCLSEEEAASLHAAIGMADLDMIDLSDDNWQWTDSPPRVDGWDSDPADVDLYDRDDVDWDGQYSSGRKRRSGRDFVMSVDSFARRHRKPRMETQEDINQRMRSVELAVKEALSARGDKQFTDQEFPPNDRSLFVDTQNPPSKLQVVSEWMRPDSIVKENGSDSRPCLFSGDANPSDVCQGRLGDCWFLSAVAVLTEVSRISEVIITPEYNEEGIYTVRFCIQGEWVPVVIDDWIPCESPGKPAFATSRKLNELWVSMVEKAYAKLHGSYEALEGGLVQDALVDLTGGAGEEIDLRSAQAQIDLASGRLWSQLLRFKQEGFLLGAGSPSGSDVHVSSSGIVQGHAYSVLQVREVDGHRLVQIRNPWANEVEWNGPWSDSSPEWTDRMKHKLKHVPQSKEGIFWMSWQDFQIHFRSIYVCRVYPREMRYSVNGQWRGYSAGGCQDYSSWHQNPQFRLRATGSDASLPIHVFITLTQGVGFSRTTPGFRNYQSSHDSQLFYIGLRILKTRGRRAAYNIFLHESVGGTDYVNSREISCEMVLDPDPKGYTIVPTTIHPGEEAPFVLSVFTKASIVLEAL

SEQ ID NO: 17 - DNA, 2058 bp, Arabidopsis thaliana
  CDS (1)..(2058)
atggaaactgaatctgatgacgccaccattaccgtcgtcaaagacatgagagttcgattagagaatcgaattcgaacccaacacgacgctcacttagatctcctttcctcactccaatccattgttcccgatatagtcccatctctcgatctctcactcaaactcatctcttctttcacgaatcgacctttcgtagctacaccaccattaccagaacccaaagttgagaagaagcatcatccgattgttaaattaggaacccaactgcaacaattacatgggcatgattcaaaatcgatgcttgttgattcgaatcagagagatgctgaagctgatgggtctagtgggagtcctatggcgcttgttagagctatggttgctgagtgtttgcttcaacgtgtaccgttttctcctacggattcttctactgtgcttaggaagcttgagaatgaccagaatgctagaccggcggagaaggcggcgttacgagatttaggcggtgaatgtggaccgattcttgcggtggagaccgcacttaaatcgatggcggaagagaatggttctgtggaattggaagagtttgaggttagtgggaagcctaggatcatggttttagctatagatagaactagattgcttaaagagttaccagagagtttccaagggaataacgaatcgaatcgggttgtcgaaacgcctaattcaattgagaatgctactgtttctggtggcgggtttggtgtttccgggtcgggtaatttcccgagacctgagatgtggggaggtgatcctaatatggggtttaggccaatgatgaatgcaccaagaggaatgcaaatgatgggaatgcatcatccaatggggataatgggtagaccaccaccatttcctttgcctttgccgttgcctgtaccgtcgaatcagaagcttagaagtgaggaagaggatttgaaagatgttgaggctcttttgagtaagaagtcttttaaggagaagcaacagtctaggacaggtgaggagttgcttgatctgattcaccgtccaaccgctaaagaagccgctactgctgctaagtttaaaagcaaaggaggatcacaggttaagtattactgtaggtatttaactaaagaggattgccgtcttcagtctggttctcacatagcctgcaacaagagacatttccgtcgactaatcgcttcacatactgacgttagcctaggagattgttcctttctcgatacttgtcgtcacatgaagacttgcaaatacgtgcattacgagctcgacatggctgacgctatgatggctggtccagataaggcattgaagcctctacgtgctgattactgttctgaagctgaacttggtgaggcacaatggattaactgtgacatccgttcttttcgaatggacattttgggaacctttggagttgttatggcggatccaccatgggacattcacatggaacttccatatggaacaatggctgatgacgaaatgcgaactcttaatgttccctcgttacagactgacggtttgattttcctctgggtcactggtcgtgctatggaactaggccgtgaatgtttggagctttggggatacaagcgagtggaagagatcatatgggtaaagacaaatcaacttcaacgtattattcgaacaggaagaacaggtcactggctcaaccatagcaaagagcattgtctggttggaatcaaaggaaatccagaagtgaacagaaacatcgacacagatgtgattgtcgcagaggttagagaaacaagccggaagccagacgagatgtatgcaatgttagagaggatcatgccaagagcaagaaagctcgagttattcgcccgtatgcacaatgctcatgccggatggctatcacttgggaaccagcttaacggtgtccgattaataaacgaaggtcttcgagctcggtttaaagcatcataccctgaaatagatgtccagccaccatcaccaccaagagcctctgcaatggaaacagacaacgaaccaatggctattgactcaatcacagcttag

SEQ ID NO: 18 - protein, 685 aa, Arabidopsis thaliana
METESDDATITVVKDMRVRLENRIRTQHDAHLDLLSSLQSIVPDIVPSLDLSLKLISSFTNRPFVATPPLPEPKVEKKHHPIVKLGTQLQQLHGHDSKSMLVDSNQRDAEADGSSGSPMALVRAMVAECLLQRVPFSPTDSSTVLRKLENDQNARPAEKAALRDLGGECGPILAVETALKSMAEENGSVELEEFEVSGKPRIMVLAIDRTRLLKELPESFQGNNESNRVVETPNSIENATVSGGGFGVSGSGNFPRPEMWGGDPNMGFRPMMNAPRGMQMMGMHHPMGIMGRPPPFPLPLPLPVPSNQKLRSEEEDLKDVEALLSKKSFKEKQQSRTGEELLDLIHRPTAKEAATAAKFKSKGGSQVKYYCRYLTKEDCRLQSGSHIACNKRHFRRLIASHTDVSLGDCSFLDTCRHMKTCKYVHYELDMADAMMAGPDKALKPLRADYCSEAELGEAQWINCDIRSFRMDILGTFGVVMADPPWDIHMELPYGTMADDEMRTLNVPSLQTDGLIFLWVTGRAMELGRECLELWGYKRVEEIIWVKTNQLQRIIRTGRTGHWLNHSKEHCLVGIKGNPEVNRNIDTDVIVAEVRETSRKPDEMYAMLERIMPRARKLELFARMHNAHAGWLSLGNQLNGVRLINEGLRARFKASYPEIDVQPPSPPRASAMETDNEPMAIDSITA

SEQ ID NO: 19 - DNA, 2397 bp, Arabidopsis thaliana
  CDS (1)..(2397)
atggatgcttcggtggtgagattttcccaatcgccggcaagagtgccaccggaatttgaaccagatatggagaagattaaacggaggctgctcaagtacggtgttgatccaacccccaaaatcctgaacaatctccgaaagaaagaaattcaaaaacacaaccgtagaaccaagcgcgaaaccgagtccgaggcggaggtgtatacggaggcgcagaaacaatcaatggaggaagaagctcgttttcaaacccttagacgggaatacaagcaattcacgaggtcaatttctggaaaaaggggcggcgatgttggtttgatggttgggaatccatgggaaggaatcgagagagtgaagctgaaggagctcgttagtggcgttcggagagaagaggttagtgctggtgaattgaagaaagagaatctaaaagagttgaagaagatacttgagaaggatcttcgttgggttctagacgacgacgttgatgtggaagaatttgatttggacaaagaatttgatcctgcgaaacggtggcgtaacgaaggagaagcagtcagagttctcgttgacagattgagtggtagagaaatcaatgagaagcattggaagtttgtgagaatgatgaatcaatcagggcttcagttcactgaagatcagatgcttaagatcgttgatcgattgggacgtaaacagagctggaaacaagcttcagctgttgttcattgggtgtattctgataaaaagcgtaaacatcttaggagcagatttgtttacaccaagcttttgtccgttcttgggtttgcgaggaggccacaggaagctcttcagatattcaatcagatgcttggtgatcgccagttatatcctgatatggcggcgtaccactgtattgctgtaacacttgggcaagcgggtttattgaaggagttgcttaaagtaatcgagcgtatgaggcagaaaccgactaaactaactaagaatttgcggcaaaagaactgggatcctgtgcttgaacctgacttggttgtatacaacgctattcttaacgcttgtgttccaacactccaatggaaggctgtttcatgggtatttgtagagttaagaaaaaatggtttgaggcctaatggagctacatatgggcttgcgatggaggttatgctggagtcagggaagtttgatcgtgttcacgatttttttaggaagatgaaaagcagtggcgaagctccaaaagcaatcacatacaaggttcttgtccgagctctctggagagaaggcaagatcgaagaagctgttgaagcagtcagagatatggaacaaaagggagttataggaacgggttcagtttactatgaattggcatgctgtctgtgcaacaacgggcgttggtgcgatgcaatgctcgaagtggggaggatgaaaagacttgaaaattgcaggccgctcgagattaccttcacaggactcatagccgcttcattgaacggtggtcatgttgatgattgcatggctatattccaatatatgaaagataaatgtgatccgaatataggaactgcgaacatgatgcttaaagtttacggaaggaatgatatgttttcagaagctaaagaattgtttgaagagattgtcagcagaaaagagactcatttagttccaaacgagtatacatacagcttcatgcttgaagcttcagctagatcactgcaatgggaatactttgaacatgtgtatcaaacgatggttctttctggttaccaaatggatcaaacaaaacatgcatcaatgctgatagaagcatcaagagccgggaagtggagtcttctagagcatgccttcgatgcagtacttgaagatggagaaatccctcacccattgttcttcactgaactgttgtgtcatgccacagctaaaggcgatttccaaagagctatcacactgattaacactgtggctctcgcatctttccagattagtgaagaagaatggactgatctttttgaggagcatcaagactggcttactcaagataatcttcataagctctcggatcatctcattgagtgtgattatgtaagtgaaccaacggtctcaaacctctcaaagtcattgaaatctcgatgtgggtcttcttcttcttcagcacaaccattgttagccgtagatgtaacaactcaaagtcagggcgaaaaaccggaggaagatttgcttctgcaagatacaacaatggaagatgataatagtgctaatggtgaagcttgggaatttacagagactgaactcgaaacgttgggtctagaagaactcgagattgatgatgatgaagaatctagcgactctgattcactttcagtttatgacattctgaaagaatgggaagagagtagtaagaaagaatga

SEQ ID NO: 20 - protein, 798 aa, Arabidopsis thaliana
MDASVVRFSQSPARVPPEFEPDMEKIKRRLLKYGVDPTPKILNNLRKKEIQKHNRRTKRETESEAEVYTEAQKQSMEEEARFQTLRREYKQFTRSISGKRGGDVGLMVGNPWEGIERVKLKELVSGVRREEVSAGELKKENLKELKKILEKDLRWVLDDDVDVEEFDLDKEFDPAKRWRNEGEAVRVLVDRLSGREINEKHWKFVRMMNQSGLQFTEDQMLKIVDRLGRKQSWKQASAVVHWVYSDKKRKHLRSRFVYTKLLSVLGFARRPQEALQIFNQMLGDRQLYPDMAAYHCIAVTLGQAGLLKELLKVIERMRQKPTKLTKNLRQKNWDPVLEPDLVVYNAILNACVPTLQWKAVSWVFVELRKNGLRPNGATYGLAMEVMLESGKFDRVHDFFRKMKSSGEAPKAITYKVLVRALWREGKIEEAVEAVRDMEQKGVIGTGSVYYELACCLCNNGRWCDAMLEVGRMKRLENCRPLEITFTGLIAASLNGGHVDDCMAIFQYMKDKCDPNIGTANMMLKVYGRNDMFSEAKELFEEIVSRKETHLVPNEYTYSFMLEASARSLQWEYFEHVYQTMVLSGYQMDQTKHASMLIEASRAGKWSLLEHAFDAVLEDGEIPHPLFFTELLCHATAKGDFQRAITLINTVALASFQISEEEWTDLFEEHQDWLTQDNLHKLSDHLIECDYVSEPTVSNLSKSLKSRCGSSSSSAQPLLAVDVTTQSQGEKPEEDLLLQDTTMEDDNSANGEAWEFTETELETLGLEELEIDDDEESSDSDSLSVYDILKEWEESSKKE

SEQ ID NO: 21 - DNA, 2511 bp, Arabidopsis thaliana
  CDS (1)..(2511)
atgatccgtggaagaaccgcgaaagtaattcccagaaatgtcatttttacgcttagtaggagttccatttctgagtctccattgatttctccgagtcgaataaaccctaaattagccggtagtttctcgttcaacatccgattattgagctatttcactgttagaaatggcttttgtcccgattgttctgtaccgagagaccctaattttgttggattgactacacaatgtcggagtattgtcaggaggttttgtagtgaaaagattggaagtagtgaatctagtggctggacagaggaggttgagtatttagatgagtcgggtagtgtactacacagtggtaagggaataagatcagtagagccggggcttgatgaccatgtaatggttggtggattgaagaagccttacatgaatgcttcttctgttgcaaagattgttgaagttgttcagaggtggaaatggggaccggagttggagactcagttagataaactccagtttgttccgaatatggttcatattacgcagtctttgaagattgttaaagaggttgatgcggcgctgagtctgtttaggtgggctaagaagcagccttggtatctgccttcggatgaatgttatgtcgtgttgtttgatgggttgaaccagggaagggattttgttgggattcagtcgttgtttgaggagatggttcaagactcgagtagtcacggtgatttgtcgtttaacgcctataaccaagtgattcagtatttggctaaagctgagaaactggaggttgctttttgttgtttcaaaaaggctcaagagtcgggatgcaaaattgatacgcagacatataataatctaatgatgttgtttctgaacaaaggtctcccgtataaggcatttgagatttatgagagcatggagaaaactgatagtttgttggatgggtcaacttatgagctgataattccaagcttggccaaatccggccgtcttgatgcagctttcaagctttttcagcaaatgaaagaaaggaaactccgaccaagctttagtgtgttttcttcgcttgttgattcaatggggaaagctggtcgattggacacatcaatgaaggtttacatggaaatgcagggatttggccataggccatcagcaactatgtttgtttccttgattgattcatatgctaaagctggtaagctggatactgctcttaggctttgggatgagatgaagaagtcaggtttcaggccaaactttggattgtacacaatgatcattgaatctcatgcaaaatcaggaaagcttgaagtagcaatgacggttttcaaagacatggagaaagctgggtttttaccgacaccatccacatattcgtgtctattggagatgcatgctggatctgggcaagtagactctgcaatgaaaatctataactccatgactaatgctgggttaaggcctggcctgagcagttatatttctcttcttacacttctggccaacaaaagacttgtcgatgttgctgggaagatactactcgagatgaaagcaatggggtattccgtagatgtctgcgctagcgatgttttgatgatatatatcaaagatgcttctgtggatcttgctctgaaatggcttaggttcatgggttcttcagggatcaaaacaaacaattttatcatcaggcagttatttgaatcatgcatgaaaaatggtctatacgattcagctaggcctttgctggagacactcgtgcattctgctggaaaagttgacttggtgctttacacttcgattctcgcccatcttgtcagatgccaagacgaagataaagagagacaattgatgtcaatcctcagcgctaccaagcataaagctcatgcctttatgtgtggtctcttcacaggtccagaacagaggaaacaaccagttctaacgtttgtcagagagttttaccaagggattgattacgaactcgaagaaggagctgctaggtactttgtgaatgtccttctcaactaccttgtcttgatgggtcaaataaaccgagctcgatgtgtctggaaagtagcgtacgagaacaaactctttccaaaagccatcgtctttgatcaacacattgcttggtctctcgatgtgagaaacttatctgtcggagctgcgctcatagccgtggttcacactctccacaggttcagaaaacgaatgctttactacggagtagtcccgagacgtataaagctagtcacaggaccgacattgaagattgtaattgctcaaatgctgagctctgttgagtcgccttttgaagtcagcaaagtcgtcttgagggcaccaggagagttggtgatggagtggttcaagaaaccgatcgtgcaacagtttcttctgaacgagatcccatcacggtctgatatattgatgcataagatgaacgtgatgttcccaagctcggctcctgagcttagatctatgtcacctcccaaaccactcatgtcatcgaaagcgttctaa

SEQ ID NO: 22 - protein, 836 aa, Arabidopsis thaliana
MIRGRTAKVIPRNVIFTLSRSSISESPLISPSRINPKLAGSFSFNIRLLSYFTVRNGFCPDCSVPRDPNFVGLTTQCRSIVRRFCSEKIGSSESSGWTEEVEYLDESGSVLHSGKGIRSVEPGLDDHVMVGGLKKPYMNASSVAKIVEVVQRWKWGPELETQLDKLQFVPNMVHITQSLKIVKEVDAALSLFRWAKKQPWYLPSDECYVVLFDGLNQGRDFVGIQSLFEEMVQDSSSHGDLSFNAYNQVIQYLAKAEKLEVAFCCFKKAQESGCKIDTQTYNNLMMLFLNKGLPYKAFEIYESMEKTDSLLDGSTYELIIPSLAKSGRLDAAFKLFQQMKERKLRPSFSVFSSLVDSMGKAGRLDTSMKVYMEMQGFGHRPSATMFVSLIDSYAKAGKLDTALRLWDEMKKSGFRPNFGLYTMIIESHAKSGKLEVAMTVFKDMEKAGFLPTPSTYSCLLEMHAGSGQVDSAMKIYNSMTNAGLRPGLSSYISLLTLLANKRLVDVAGKILLEMKAMGYSVDVCASDVLMIYIKDASVDLALKWLRFMGSSGIKTNNFIIRQLFESCMKNGLYDSARPLLETLVHSAGKVDLVLYTSILAHLVRCQDEDKERQLMSILSATKHKAHAFMCGLFTGPEQRKQPVLTFVREFYQGIDYELEEGAARYFVNVLLNYLVLMGQINRARCVWKVAYENKLFPKAIVFDQHIAWSLDVRNLSVGAALIAVVHTLHRFRKRMLYYGVVPRRIKLVTGPTLKIVIAQMLSSVESPFEVSKVVLRAPGELVMEWFKKPIVQQFLLNEIPSRSDILMHKMNVMFPSSAPELRSMSPPKPLMSSKAF

SEQ ID NO: 23 - DNA, 2922 bp, Arabidopsis thaliana
  CDS (1)..(2922)
atgagctctcatcatcaaatcctccaaattcgttctgacccatttgttctatcacactgttgccgccacacacgcctcaccagctctcttacgcttcaatctcctctaaagcagcccttttcatgtctccctttccgttggcgccgctcttatcgcggcggagtcaggagctcaacgacggagactcatggtagcaaaaaagaagctttggtgtcggaaacggcgactacatcgattgaattgaaaagggtctatccatttcacgaaattgaacccaagtggcaacgttattgggaagataaccgaattttccggacaccagacgatgttgatacttcgaaacctaagttctacgtcctcgacatgtttccatatccaagtggagcaggattacatgtgggacatccattgggttatacagcaactgatattctagcgaggcttaggcgtatgcaaggttacaatgtgttgcatcctatgggttgggatgcttttggattgcctgctgaacaatatgctattgagacaggaacccatccgaaaactacgacattgaagaacattgaccgctttcgattgcagctcaaatcattgggcttctcatacgattgggaccgtgaactttctacaacagagccagactactacaaatggacacaatggatctttcttcagctttataagaaaggtttggcatatcaggctgaagtaccggtcaattggtgcccggctcttggtactgttttggccaatgaagaagtggtggatggtgttagtgagcgtggtggccacccggttataagaaagccgatgaggcaatggatgctgaagattactgcgtacgctgatcgtcttctagaagatttggacgagcttgagtggcctgaaagtataaaggaaatgcaaagaaactggataggaagatccgaaggagctgagttgaacttttcaattcttgatggggaaggccgagaaactgacaaagagattacagtgtacaccacgaggccagatacactttttggagcaacttacatggttgtggcaccagagcatcagttgttgtcttactttgtaaccgcagaacagaaacagcaagttgaggaatacaaagatttcgcttcgaggaaaagtgatcttgagagaacagaacttcagaaggacaagacgggcgtatttacgggatgttatgcgaaaaatccagctaatggagatgctattcccatatgggtagctgattatgttctagctagttatggaaccggagcaatcatggcggtcccagctcatgatactcgggataatgagtttgcgttgaagtataacattcccatcaagtgggtagtgagaaatgaggcaaattcaagtgatgatgctaagcaggtttatcccggattgggcattattgaaaattcttcaactttagaaacaggacttgacataaatcaactatctagcaaagaagctgctttaaaagttattgaatgggctgagagaactggcaatggaaagaaaaaggtaaattacaagttgagggattggctatttgcacggcagcgttactggggtgaacctatcccgattttaattttggatgagtctggtgaaactattgctatttcagaatctgaactgcccctcactttgcctgagttaaatgattttacgcccactggaacaggggaaccaccactgtcaaaagcagtttcatgggtgaacactgtagatccttcaacaggaaaacctgccaaaagagaaacaagcactatgccacaatgggctgggtcttgctggtactatctgagattcatggacccgaaaaaccctgaagcattagttgacaaggaaaaagaaaaatactggagcccagtagatgtatatgttggtggtgctgagcatgctgttttacatttactatactcgaggttttggcacaaggtactttatgacattggtgttgtgtctacgaaagaaccattcaaatgtgtcataaatcaaggaatcatccttggggaagtccaatatacagcttggaaagatcaagaaggaaattatgtatctgcagatactgaagaacggttaaatgaacatcaacaagtgacaatcccggaagaaaaagttatcaagtccggagatcattttgttctgaaggaggatcctagcattcgtctgattccacgcgtttataaaatgagcaaaagtagaggaaatgtcgtaaatcctgatgatgttgtattagagtatggtgcagattctctacgtttgtatgagatgttcatgggaccatttagggactcaaaaacttggaacacgagtgggattgaaggtgtgcatcgtttcttggcaagaacttggagactagtcattggtttgcctcagtctgatggttctttcaaagatggaaccttagtaactgatgacgaaccaacccttgaacaactccgaactcttcataaatgcattgcaaaggtgacagaggaaatcgagagtacacgattcaacacgggaatttcagggatgatggagtttgttaatgcggcatataagtggaataatcaacctagaggaatcattgagccttttgttcttttgctctcaccttacgcgcctcacatggctgaagagctttggtcgcggttaggccatccaaattctctggcctatgaaagcttccctaaggcaaacccggattacttaaagaacacaactattgttcttccagttcagataaacggcaagacaagaggcaccatagaggttgaagaaggatgctctgaagatgatgcttttgttctagcttcacaggacgacaaactcaggaaatatctagatggacaatcaatcaagaagcgaatctatgtgcctgggaagatcctcaacgttatcctagaccggactaatgtcaaggtcactacaaagtag

SEQ ID NO: 24 - protein, 973 aa, Arabidopsis thaliana
MSSHHQILQIRSDPFVLSHCCRHTRLTSSLTLQSPLKQPFSCLPFRWRRSYRGGVRSSTTETHGSKKEALVSETATTSIELKRVYPFHEIEPKWQRYWEDNRIFRTPDDVDTSKPKFYVLDMFPYPSGAGLHVGHPLGYTATDILARLRRMQGYNVLHPMGWDAFGLPAEQYAIETGTHPKTTTLKNIDRFRLQLKSLGFSYDWDRELSTTEPDYYKWTQWIFLQLYKKGLAYQAEVPVNWCPALGTVLANEEVVDGVSERGGHPVIRKPMRQWMLKITAYADRLLEDLDELEWPESIKEMQRNWIGRSEGAELNFSILDGEGRETDKEITVYTTRPDTLFGATYMVVAPEHQLLSYFVTAEQKQQVEEYKDFASRKSDLERTELQKDKTGVFTGCYAKNPANGDAIPIWVADYVLASYGTGAIMAVPAHDTRDNEFALKYNIPIKWVVRNEANSSDDAKQVYPGLGIIENSSTLETGLDINQLSSKEAALKVIEWAERTGNGKKKVNYKLRDWLFARQRYWGEPIPILILDESGETIAISESELPLTLPELNDFTPTGTGEPPLSKAVSWVNTVDPSTGKPAKRETSTMPQWAGSCWYYLRFMDPKNPEALVDKEKEKYWSPVDVYVGGAEHAVLHLLYSRFWHKVLYDIGVVSTKEPFKCVINQGIILGEVQYTAWKDQEGNYVSADTEERLNEHQQVTIPEEKVIKSGDHFVLKEDPSIRLIPRVYKMSKSRGNVVNPDDVVLEYGADSLRLYEMFMGPFRDSKTWNTSGIEGVHRFLARTWRLVIGLPQSDGSFKDGTLVTDDEPTLEQLRTLHKCIAKVTEEIESTRFNTGISGMMEFVNAAYKWNNQPRGIIEPFVLLLSPYAPHMAEELWSRLGHPNSLAYESFPKANPDYLKNTTIVLPVQINGKTRGTIEVEEGCSEDDAFVLASQDDKLRKYLDGQSIKKRIYVPGKILNVILDRTNVKVTTK

SEQ ID NO: 25 - DNA, 774 bp, Arabidopsis thaliana
  CDS (1)..(774)
atgtcgatgtcgaagagttcaaagatgcttcagttcatcaactacaggatgcgagtgacgatccaagacggaagacagctcgttgggaagttcatggcgtttgaccgtcacatgaacctcgttctcggcgattgcgaggagtttcgtaagcttccaccagctaaagggaagaagatcaacgaagagcgtgaagatcgccgtacgctaggtttagtgttgcttagaggtgaagaagttatctcaatgactgtcgaaggaccacctcctcctgaagaatctcgtgctaaagctggctctgcagctgctgttgctggtccaggaattggccgtgccgctgggcgtggagttcctactggtccgttagttcaagctcagcctggtttatctggtcctgttcgtggtgttggtggacctgctccgggaatgatgcagcctcagatctctcgtccaccacagctttcagctcctccgattattcgacctccgggacagatgttgccaccgcctccttttggtggtcaaggtcctcctatgggaagaggtcctccaccgccttatggtatgaggccaccgccacagcagttttctggaccaccaccgcctcagtatgggcaaaggccaatgattcctcctcctggtggtatgatgagaggacctcctcctccacctcatggaatgcaaggacctcctccgcctcgccctggaatgcctcctgctcctggtggttttgctccaccgcgtcctggcatgccaccacataatcagcagcagtga

SEQ ID NO: 26 - protein, 257 aa, Arabidopsis thaliana
MSMSKSSKMLQFINYRMRVTIQDGRQLVGKFMAFDRHMNLVLGDCEEFRKLPPAKGKKINEEREDRRTLGLVLLRGEEVISMTVEGPPPPEESRAKAGSAAAVAGPGIGRAAGRGVPTGPLVQAQPGLSGPVRGVGGPAPGMMQPQISRPPQLSAPPIIRPPGQMLPPPPFGGQGPPMGRGPPPPYGMRPPPQQFSGPPPPQYGQRPMIPPPGGMMRGPPPPPHGMQGPPPPRPGMPPAPGGFAPPRPGMPPHNQQQ

SEQ ID NO: 27 - DNA, 1311 bp, Arabidopsis thaliana
  CDS (1)..(1311)
atggcgtcgtcttctctcacttcgaaatccattctcggatccaccaaactcggttcttcttctcttccctcggagctccgtcgtctctcttctcccgccgttcagatctctctccgtacccaaaccaggaagaacttccagatacaagctactggaagttcatatgggactcattttcgagtttcaacttttggagaatcacatggaggaggagttggttgtatcattgatggttgtcctcctcgtattccacttactgaatctgatttgcaattcgatctcgatagaaggaggcctggtcagagcaggatcacaactcctagaaaagagactgatacttgccggatatcgtctggagtctctgaaggaatgacgacaggaacacctatccatgtgtttgtaccaaacacagatcagagaggacttgattacagtgaaatgtcggttgcctatagaccatcgcatgctgatgcaacttatgacatgaagtatggtgtcagatcagtgcagggtggaggaagatcttcagctagagagaccattggaagagttgctcctggagctttggccaagaaaattttgaagcaatttgcaggaactgagattcttgcctatgtctcgcaagttcaccatgttgtacttccagaagaattggtagaccacgagaatttaacactcgaacagatagaaaataacattgtcagatgccctaatcccgagtatgcggaaaagatgatagctgcgattgatgctgtcaggacaaaagggaactctgttggtggtgttgtgacctgcattgttcggaatgctccacgtgggcttggtacaccggttttcgataaacttgaagcagaactggcaaaagcttgtatgtcgctacctgcaactaagggatttgagtttggaagcggctttgcaggtacctttttgactggtcttgaacacaatgatgagttctataccgatgaaaatggaagaatacgtaccagaaccaaccgatctggtggaattcagggagggatctcaaatggtgaaataataaacatgagagtagccttcaagccaacatcaacaattggaaggaagcaaaatacggtaaccagagacaaggtagaaaccgaaatgattgcgcgtggtcgtcatgatccttgtgttgttccacgagctgtgccaatggtggaagcaatggtggctctagttcttgtggatcaattgatggcgcaatacgcacaatgccatttgtttccaataaatccagagttgcaggaacctctccagatagagcagccgcaaaatgctactgctttgtaa

SEQ ID NO: 28 - protein, 436 aa, Arabidopsis thaliana
MASSSLTSKSILGSTKLGSSSLPSELRRLSSPAVQISLRTQTRKNFQIQATGSSYGTHFRVSTFGESHGGGVGCIIDGCPPRIPLTESDLQFDLDRRRPGQSRITTPRKETDTCRISSGVSEGMTTGTPIHVFVPNTDQRGLDYSEMSVAYRPSHADATYDMKYGVRSVQGGGRSSARETIGRVAPGALAKKILKQFAGTEILAYVSQVHHVVLPEELVDHENLTLEQIENNIVRCPNPEYAEKMIAAIDAVRTKGNSVGGVVTCIVRNAPRGLGTPVFDKLEAELAKACMSLPATKGFEFGSGFAGTFLTGLEHNDEFYTDENGRIRTRTNRSGGIQGGISNGEIINMRVAFKPTSTIGRKQNTVTRDKVETEMIARGRHDPCVVPRAVPMVEAMVALVLVDQLMAQYAQCHLFPINPELQEPLQIEQPQNATAL

SEQ ID NO: 29 - DNA, 1038 bp, Arabidopsis thaliana
  CDS (1)..(1038)
atgctttcgttatcatgctcttcttcttcttcttcgttgcttcctccgagtttacactaccacggttcttcttctgttcagtccatcgttgtaccaagaaggagtcttatctcgtttcgtcggaaagtctcttgctgttgcatagctccacctcagaacttggacaacgatgccaccaaattcgatagtcttaccaagtctggaggaggtatgtgtaaagagcgagggcttgagaatgattctgatgttcttattgaatgtagagatgtctataaatcgtttggggagaaacatatcttgaaaggtgttagctttaagattagacatggtgaagctgttggggtgattggtccttctggaactggaaaatcaacaattttaaagattatggctggtcttcttgctccagacaagggagaagtttatatacgaggaaaaaaacgagctggtttgataagtgatgaggaaatatcaggacttcgtattggcctggtatttcagagtgcagctctctttgattcactatcagttcgtgaaaatgttggttttctactttatgaaagatcaaaaatgtccgagaatcaaatatctgagcttgtgacacaaaccttggcagctgttggtttgaagggggttgagaatcgattaccttctgagctatctggtggaatgaagaaaagggttgctttagctcgttcactaatttttgatacaacaaaagaggtcatagagccagaggtgcttttgtacgatgagccaactgctggacttgatccaattgcatcaactgtagttgaagatcttatacggtctgttcacatgacagacgaagatgcagttggaaaacctggaaaaattgcgtcttatcttgttgttacccatcaacatagcaccattcaaagagctgtagacaggttattgtttctgtatgaaggaaagatcgtttggcaaggaatgacacatgaattcacaacctcaactaatccaatagttcaacagtttgctacaggcagcctcgatggaccaatcagatactag

SEQ ID NO: 30 - protein, 345 aa, Arabidopsis thaliana
MLSLSCSSSSSSLLPPSLHYHGSSSVQSIVVPRRSLISFRRKVSCCCIAPPQNLDNDATKFDSLTKSGGGMCKERGLENDSDVLIECRDVYKSFGEKHILKGVSFKIRHGEAVGVIGPSGTGKSTILKIMAGLLAPDKGEVYIRGKKRAGLISDEEISGLRIGLVFQSAALFDSLSVRENVGFLLYERSKMSENQISELVTQTLAAVGLKGVENRLPSELSGGMKKRVALARSLIFDTTKEVIEPEVLLYDEPTAGLDPIASTVVEDLIRSVHMTDEDAVGKPGKIASYLVVTHQHSTIQRAVDRLLFLYEGKIVWQGMTHEFTTSTNPIVQQFATGSLDGPIRY

SEQ ID NO: 31 - DNA, 4035 bp, Arabidopsis thaliana
  CDS (1)..(4035)
atggcgtcttctcctggtaatactaaccctcacaatactcctccgtttgatctcggtattctcttcaaaccttcatccaatccttatccgccaccggcggcatcttatcctccgcctacaggtccttttcttcacaaccagtacgatcagcagcactacgctccgcctggtatctccgctcaaccatctccggtgactcagcagcaacaggatgtgtcttcctcttccgccgcgactaacttgcatcctcagagaacgctgtcttatcccacgccacctctcaatctacaatccccccgttccaatcacaatcccggtacgcacatcctcgctctccttaacaataccaacaacggagcccccgtggctaaccaagagccgtcgcatcagctcccagtagtcaatcacaacgagatcgctcgctcttttcccggcggttcaggtcctattcgtgtaccgagctgtaaattgcccaagggaaggcgattgattggtgaacacgctgtgtacgatgttgatgtgagattacaaggtgagattcagccgcagctggaggtgactccgattaccaaatacgggtcggatcctcagctcgtagtgggtaggcaaatcgccgtgaataaggtttacatttgctatggattgaaaggaggaaacattagggttctcaatatcaacacagcattgaggtctctgttccgtggccattcacagagagtgacggatatggctttctttgccgaggatgttgatatgttggccagtgttagcctagatggaaaagtttttgtgtggaaaatttctgaagggtctgagggagaggatcagccccaaataactgggaagatagtacttgctcttcagatactaggagaggaagacaccaaacatccacgcgtttgttggcactgccacaaacaggaaattttggtagtttcaattggtaaacatgtactgcgtattgatactacaaaagttggcagaggtgaagtattctctgctgaggctcctctccagtgtccccttgataagctgatcgatggtgttcagattgttggtaagcatgatggagaagtgacagatttgtcaatgtgccaatggatgaccacgcgtctggtttcttcttcagttgatggcacgattaagatatggcaagatcgtaaggcacaaccacttgtagttttgaggcctcatgatggacatccagtcagttcagccacgtttgtgacatctcctgagagacctgatcacatcatacttatcacggggggtcctctaaatcgagaaatgaaaatctgggtctctgctggggaagaagggtggcttttaccagctgatgctgaatcatggaggtgtacccagactcttgacttgaaaagttcaaccgagccacgagctgaagaggcattttttaaccaagtcatagcattatctgaagcaggcttgcttttacttgcaaatgcaaaaaggaacgccttatatgctgtgcatttggactatggttcttctccagttggtacgcggatggattacctatcagagtttacagtcactatgcccatattgagtttcattgggacaaatgatcctccagaagaacccattgttaaggtttattgtgttcagactctagcaatccagcagtatacattagacttgtgcttatgcttgccaccacctatagaaaatatgggtttggagaagtcagattctagtgtatcacgagaggcaaatcttgttgaaggcatgtcagaaccatctggacttaaacctactgacttaccttcagttgattcagtgccaaaaccttcgattatagtgaatagatcagaaagtgctaataagttgagttttccatcagcggaagccacatcacaagcaattgttccacccaacggtgaacctaaaacttctggcctgccatctcaaaccagtggtgcaggttctgcgtatgctactttaccccaacttcctctcagtcccagactatcaagtaaactttctggctatcacactcctgtagaggcgattgagccagtaatacctcatcatgagcttggtggcaaaacaccttctgctgattactctgttgataggcaaatggatgctgttggagaaagaaatttggacgtgtcatccgtagaagaaatctcaagaagcaaggactcaaatgttacgcctgatgatgatgtgtctgggatgcgaagcccatcagcttttttcaaacaccccactcatcttgtaactccttcagagatattgatgggcgtttcttccgctgaagcctccattactactgaagacaggagagatagggatgcaaatattcaggacgtgaataatgatccaagagacaccgaagtagaggtgaaagaaataagtgaagcaaggtcaacacagaatggtgaaatcaatgatcatgacgaaactgagaattgcacttcagaaaatagagaaaaagtcttctgctcacaggtttcaaatctcagcactgagatggcaagagactgttatcctagtactgagggaactttcattccaggggaatctaaggcatatggacaacctataaaggctggagatgaaagtggtgttgactcgagaggtgggcctgcaaagcttcttaaaggaaagaagcagaaggccaaaaattcacagggtcccggtttgtcatctacatcctcaaatgttgctaatctggctgactccttcaatgagcaaagtcagagtttaagtcatcccatgacagatttacttcctcagttgttagcaatgcaagaaacgatgaatcaggtgatggcttcgcagaaggagatgcagagacaactatcaaatgctgccactggccctatcggaaaagaaagtaaaagactagaagttgcgttagggagaatgattgagaaatccagcaagtcaaatgctgatgctctatgggcccgcatccaggaggagactgttaagaatgaaaaggcattacgtgaccatgcccagcaaattgtgaatgcaacgacaaacttcatgagcaaggagttaaatgccatgtttgagaaaacgataaagaaagaattggctgcaattggtccagccctagcacgttcagtagtaccagttattgaaaaaactgtatcttctgcaatcacagagtcctttcagagaggaataggtgacaaagcagttaatcagcttgacaaatctgttaatataaagcttgaagcaaccgtagctaggcaaattcaagcccaatttcagacctctggcaagcaagccctccaggaaggtcttaggtcaagtgtggagtcctcagtcataccttcctttgagaaggcatgcaaggccatgtttgaccaaatagactcagccttccagaaaggtattgctgagcatacaaatgcagcccagcaacggtttgactctggacactcccagcttgctcatactctaaaggaatcaattacttctgcgtcgtcagttgctcaagccttaagtcgtgagttagccgagacccaaaggaatctcttagctctcgcagctgctggagcaaattctggtgggtctaattccttggttactcaactaagtggcggacctttgggtgctcttcttgaaaaggttgaagcacctatggacccaacaacagaactatcgaggttgatatctgaacggaagtacgaagaatctttcacttcggccctacagagaagcgatgtctctatagtatcatggctttgctcacaggtggatctacgtggattactggcgatgaatccgcttccactgagccaaggcgtgcttctttcactgctgcagcagctagcctgtgacattagcaaggacacatcccgtaagctggcttggatgactgatgtggttgcagccataaacccatcagaccagatgattgcggtccacgctcggccaatctttgaacaggtatatcagattctgcaccatcaccgcaacgcaccgggaagcgatgtctcagccatcagactgataatgcacgtgatcaactccatgcttatgggttgcaaatga

SEQ ID NO: 32 - protein, 1344 aa, Arabidopsis thaliana
MASSPGNTNPHNTPPFDLGILFKPSSNPYPPPAASYPPPTGPFLHNQYDQQHYAPPGISAQPSPVTQQQQDVSSSSAATNLHPQRTLSYPTPPLNLQSPRSNHNPGTHILALLNNTNNGAPVANQEPSHQLPVVNHNEIARSFPGGSGPIRVPSCKLPKGRRLIGEHAVYDVDVRLQGEIQPQLEVTPITKYGSDPQLVVGRQIAVNKVYICYGLKGGNIRVLNINTALRSLFRGHSQRVTDMAFFAEDVDMLASVSLDGKVFVWKISEGSEGEDQPQITGKIVLALQILGEEDTKHPRVCWHCHKQEILVVSIGKHVLRIDTTKVGRGEVFSAEAPLQCPLDKLIDGVQIVGKHDGEVTDLSMCQWMTTRLVSSSVDGTIKIWQDRKAQPLVVLRPHDGHPVSSATFVTSPERPDHIILITGGPLNREMKIWVSAGEEGWLLPADAESWRCTQTLDLKSSTEPRAEEAFFNQVIALSEAGLLLLANAKRNALYAVHLDYGSSPVGTRMDYLSEFTVTMPILSFIGTNDPPEEPIVKVYCVQTLAIQQYTLDLCLCLPPPIENMGLEKSDSSVSREANLVEGMSEPSGLKPTDLPSVDSVPKPSIIVNRSESANKLSFPSAEATSQAIVPPNGEPKTSGLPSQTSGAGSAYATLPQLPLSPRLSSKLSGYHTPVEAIEPVIPHHELGGKTPSADYSVDRQMDAVGERNLDVSSVEEISRSKDSNVTPDDDVSGMRSPSAFFKHPTHLVTPSEILMGVSSAEASITTEDRRDRDANIQDVNNDPRDTEVEVKEISEARSTQNGEINDHDETENCTSENREKVFCSQVSNLSTEMARDCYPSTEGTFIPGESKAYGQPIKAGDESGVDSRGGPAKLLKGKKQKAKNSQGPGLSSTSSNVANLADSFNEQSQSLSHPMTDLLPQLLAMQETMNQVMASQKEMQRQLSNAATGPIGKESKRLEVALGRMIEKSSKSNADALWARIQEETVKNEKALRDHAQQIVNATTNFMSKELNAMFEKTIKKELAAIGPALARSVVPVIEKTVSSAITESFQRGIGDKAVNQLDKSVNIKLEATVARQIQAQFQTSGKQALQEGLRSSVESSVIPSFEKACKAMFDQIDSAFQKGIAEHTNAAQQRFDSGHSQLAHTLKESITSASSVAQALSRELAETQRNLLALAAAGANSGGSNSLVTQLSGGPLGALLEKVEAPMDPTTELSRLISERKYEESFTSALQRSDVSIVSWLCSQVDLRGLLAMNPLPLSQGVLLSLLQQLACDISKDTSRKLAWMTDVVAAINPSDQMIAVHARPIFEQVYQILHHHRNAPGSDVSAIRLIMHVINSMLMGCK

SEQ ID NO: 33 - DNA, 3036 bp, Arabidopsis thaliana
  CDS (1)..(3036)
atgttgcttccactgtttcaccagcaaccattgatacttgccaaaacctttcccgatcgaattttcccgccattccttgttcccaacaccctagtttcgcgacgcaatgtcagtcgagcgaactccggcatcttctgtagttctgcttccggtcgaaagactcttccccaatcggctatccagagaatcgctgagaagcttcgtagccttggattcgtggaagagaagcatgattcaccgactaggagaatcacgggagaagaatctggtaagaattccccaggagagatattcgttccgttgcctaagcagcttccgattcaccgggtcgggcacacgattgacacaagctggagcacgcctagttacccggttccgaagcccgggtcgggtactgccatttcaaggtaccatgagttgaagagagtgtggaagaaggaaacggagatggagaggaagaaggaggagaaggttccgtcgttggcggaattgacattgccgccggcggagctgaggcggttgaggacggtggggattaggttgacgaagaagctgaagattggcaaggctgggattacggaagggattgtgaatgggattcacgagagatggaggacgacggaggttgtgaagattttctgcgaagatatttccagaatgaacatgaaacggacccatgacgtcttggagacaaaaactggaggcttagtcatttggaggtctggaagcaaaatattgttgtatagaggggtaaattatcagtacccttattttgtatctgatcgggatttggcacatgaagctgcttcaggagcatcatctatggatcaaggagtggttgatagtagagagaaacagagtattgcagaatcttctgccccgagtattactaataaaatggttaaaccgatgttgacgcaaggcgttggttctccggataaggttaggtttcaattacccggggaagtacagcttgtagaagaagcagaccgtttgttggaaggattgggtccaagatttactgactggtgggcatatgatccccttcccgtagatggtgatcttctgccagccgtagtacctgactacaggagaccatttcgccttcttccatatggcgtcagtccaaaactaactgatgatgaaatgaccacaataaggagacttggtagaccactcccttgtcattttgctttaggtaggaatagaaatttgcagggactagctgttgcaattgtcaagctttgggagaaatgtgagcttgccaagatagcagtgaagagaggagttcagaacactaatagtgagctgatggccgaagagttaaagtggttgactggagggactctgatatctcgggataaagatttcattgtcctgtacagaggaaaggatttcctcccatctgcagtttcttctgcaatagaagagaggagaaggcaaacaatgataatggagaattctagtgtgcatggtaataagctgactgaaaacgaagaggaaataaaacctcgggctgttaaagaggatattgaactagaagctaaggatcagaaggatcatatacaaacccatcagatgaaatcaagacagcgaaattctcctgaggcaatccttgaaaaaactagtatgaagttgtccatggcgttggaaaagaaagcaaatgcagaaaaagtcctggcggatctggagaatagagagagtcctcaactttctgatattgacaaggagggtattactaatgatgaaaagtacatgctccggaaaattggcttgaaaatgaagcctttccttttactaggtagaagaggcgtctttgatggaacaatagagaacatgcatcttcactggaagtatagggaacttgtgaagatcatttgtaatgaatatagcattgaagctgctcacaaagtagcagaaatcttggaagcagaaagtggtggcatactagttgctgtggagatggttagtaagggctatgcaatcattgtgtatcgtggaaagaattatgagcggcctcaatgtctacgacctcaaacacttctcagtaagagagaggcgctgaagcggtctgtggaggcacaacgtcgaaagtcattgaagctacatgtgctgaaactttccaacaatatcgaagaattgaaccgtcaattggtcgaggacagcgcaaccaatgaaacttggtcagatggggaatcaagtaacatgatggtcgaagaagaaacagaaaatcagcatactgaacctgagaaagcaagagaaaaaattgaacttggctattcatctgacttatcagttccttcctctggtgaagaaaactgggaggatgacagcgaaggtgaagtagacccattaactacgagcagtcaggagtatcaagaagatgaatctgaatctgcctcgtctcagagacacgagggtaactctctcgattcaactgcaaatctgtctgtgtttgcggaaaccggttccgccaatgcttcttcatttcatgatagatcactgccgcacaacagcttcctgaatgccaatcgaaagttaccaggatcatcaactggaagtggctcacaaatctcagctctgagagagaggaaaagcgaaaatgatgggttggtcacagatctgtccaatagagaaaggctcattctgagaaaacaagcccttaagatgaaaaagcgaccaccctttgcagtaggaagaagcaacgttgttacaggtttagcaagaacactgaagatgcattttcagaaaaaccctctagcaatcgtaaacgtcaaaggaagagcaaatggaacatctgtgcaagaagtcatcgcaaagctaaaggaagaaacaggagctcttctggtgtcacaggagccaagtaaagtcatactttaccgcggatggggagcggaagaagaaatgaaaagcttctacccaaataacaatgtcaaaagctctataaatctaccctctacgagaagctttgttgatgatcctcctcatgtgtctcctgcactcatcgaagccataagacttgaatgtggtttgtaa

SEQ ID NO: 34 - protein, 1011 aa, Arabidopsis thaliana
MLLPLFHQQPLILAKTFPDRIFPPFLVPNTLVSRRNVSRANSGIFCSSASGRKTLPQSAIQRIAEKLRSLGFVEEKHDSPTRRITGEESGKNSPGEIFVPLPKQLPIHRVGHTIDTSWSTPSYPVPKPGSGTAISRYHELKRVWKKETEMERKKEEKVPSLAELTLPPAELRRLRTVGIRLTKKLKIGKAGITEGIVNGIHERWRTTEVVKIFCEDISRMNMKRTHDVLETKTGGLVIWRSGSKILLYRGVNYQYPYFVSDRDLAHEAASGASSMDQGVVDSREKQSIAESSAPSITNKMVKPMLTQGVGSPDKVRFQLPGEVQLVEEADRLLEGLGPRFTDWWAYDPLPVDGDLLPAVVPDYRRPFRLLPYGVSPKLTDDEMTTIRRLGRPLPCHFALGRNRNLQGLAVAIVKLWEKCELAKIAVKRGVQNTNSELMAEELKWLTGGTLISRDKDFIVLYRGKDFLPSAVSSAIEERRRQTMIMENSSVHGNKLTENEEEIKPRAVKEDIELEAKDQKDHIQTHQMKSRQRNSPEAILEKTSMKLSMALEKKANAEKVLADLENRESPQLSDIDKEGITNDEKYMLRKIGLKMKPFLLLGRRGVFDGTIENMHLHWKYRELVKIICNEYSIEAAHKVAEILEAESGGILVAVEMVSKGYAIIVYRGKNYERPQCLRPQTLLSKREALKRSVEAQRRKSLKLHVLKLSNNIEELNRQLVEDSATNETWSDGESSNMMVEEETENQHTEPEKAREKIELGYSSDLSVPSSGEENWEDDSEGEVDPLTTSSQEYQEDESESASSQRHEGNSLDSTANLSVFAETGSANASSFHDRSLPHNSFLNANRKLPGSSTGSGSQISALRERKSENDGLVTDLSNRERLILRKQALKMKKRPPFAVGRSNVVTGLARTLKMHFQKNPLAIVNVKGRANGTSVQEVIAKLKEETGALLVSQEPSKVILYRGWGAEEEMKSFYPNNNVKSSINLPSTRSFVDDPPHVSPALIEAIRLECGL

SEQ ID NO: 35 - DNA, 654 bp, Arabidopsis thaliana
  CDS (1)..(654)
atggcgaattcaatggctacactttctagaagactctatcgatctcttctatcgaaccctagaatttctcaagcttccatgtctttctgtaccaacaacatcacttccccagaagactccgactttgacgagttagaatcgcccattgaaccaaaggcctcagatccagtttctcgattttccggagaagaacgcgttatggaagagcgtccactcgaaaatggccttgactctggcattttcaaggcaatattggtggggcaagtgggtcagcttcctctgcagaagaagcttaagagtggtagaactgtcactctcttctctgtaggaactggtgggatcaggaacaaccggagaccgcttattaacgaagatccgagagagtatgcgagccggtctgcggtgcagtggcaccgtgtctctgtttaccctgaacgcttggcagatcttgtgttgaagaatgttgaacccggtactgttatctacttagagggtaatctggagacgaagatattcactgatcctgtcactggtttggttcgacgtataagagaagtagcaattcgtagaaacggccgagttgtgtttttgggaaaagccggtgatatgcagcaaccgagttctgctgagcttagaggcgttggctactactga

SEQ ID NO: 36 - protein, 217 aa, Arabidopsis thaliana
MANSMATLSRRLYRSLLSNPRISQASMSFCTNNITSPEDSDFDELESPIEPKASDPVSRFSGEERVMEERPLENGLDSGIFKAILVGQVGQLPLQKKLKSGRTVTLFSVGTGGIRNNRRPLINEDPREYASRSAVQWHRVSVYPERLADLVLKNVEPGTVIYLEGNLETKIFTDPVTGLVRRIREVAIRRNGRVVFLGKAGDMQQPSSAELRGVGYY

SEQ ID NO: 37 - DNA, 1320 bp, Arabidopsis thaliana
  CDS (1)..(1320)
atggcttcagatgctgacatggaggactatggattcgagtattctgatgaggagcaagaggaacaagatgttgacattgagaaccagtattataactctaaaggtatggttgagactgaacctgaagaagcactttctggatttgctgaggttgttcagatggaacctgagaaagctgactggggtttcaaagctcttaagcagactgtgaagatctattatcgtctaggtaaatacaaagaaatgatggaagcctatacagagatgcttacatatatcaagtcagcagtcaccaggaattacagcgaaaaatgtataaacaatatcatggatttcgtctctggatctgctagccagaacactggcctgctgcaagagttctatcagaccactttgaaagcccttgaagaggctaagaatgagagactctggttcaagacaaatcttaaactctgcaacatctggtttgacattggtgaatacagacggatgactaagattctgaaggaactccataagtcttgtcaaaaggaagatggaactgatgatcagaagaaaggaagtcagctgcttgaggtttatgcaattgaaattcagatttacactgaaacgaaggacaacaaaaagcttaagcaactataccataaggcacttgccatcaagtctgccatacctcatcctaggatcatgggtataatccgcgagtgtggtggaaaaatgcacatggcagaacgtcagtgggaagaagcagcgacagacttctttgaggcttttaaaaattacgatgaagctggcaaccaaagacgtatacagtgcctgaagtatcttgttctggcgaatatgctgatggagtcagaagtgaatccgtttgatggccaagaggcaaagccgtacaaaaacgaccctgagattttggcaatgacaaatttgatcgcagcatatcaacgaaatgagatcatagagtttgagagaatactaaagagtaaccggaggacgataatggatgatccattcatcagaaactacatggaagatctgctgaagaaagtgagaacacaagtgttactgaagctgattaagccatacacaaagatagggattccgttcatatcaaaggaactgaacgtgccagagacggatgtgacggagttactagtgtcgctgatactcgacagcagaatcgatggtcacattgatgaaatgaaccgttacttactgagaggcgacagtggcaatggaaggaagttgcataaggcggttgataaatggaactcacagctcaagtctctttcttcaaatatcaccagccgagtctgttaa

SEQ ID NO: 38 - protein, 439 aa, Arabidopsis thaliana
MASDADMEDYGFEYSDEEQEEQDVDIENQYYNSKGMVETEPEEALSGFAEVVQMEPEKADWGFKALKQTVKIYYRLGKYKEMMEAYTEMLTYIKSAVTRNYSEKCINNIMDFVSGSASQNTGLLQEFYQTTLKALEEAKNERLWFKTNLKLCNIWFDIGEYRRMTKILKELHKSCQKEDGTDDQKKGSQLLEVYAIEIQIYTETKDNKKLKQLYHKALAIKSAIPHPRIMGIIRECGGKMHMAERQWEEAATDFFEAFKNYDEAGNQRRIQCLKYLVLANMLMESEVNPFDGQEAKPYKNDPEILAMTNLIAAYQRNEIIEFERILKSNRRTIMDDPFIRNYMEDLLKKVRTQVLLKLIKPYTKIGIPFISKELNVPETDVTELLVSLILDSRIDGHIDEMNRYLLRGDSGNGRKLHKAVDKWNSQLKSLSSNITSRVC

SEQ ID NO: 39 - DNA, 1158 bp, Arabidopsis thaliana
  CDS (1)..(1158)
atgggtttcaaactcaactctcttttccttcttctttcgcttctcatactcacaatcttgtccggagccgaccaagccttggcttacgtcaagaaaccacatgtgagccaacgcaacaaaacggcgttagcagccgtcgccggaagaggaggaggaaaagagatgttgaaagggaggaagcagacgagtggttgtaacttgtttcaagggagatgggttttcgatgcttcttaccctttctacgattcatccacgtgccctttcatcgacggtgagttcgactgtctcaaattcggccgaccagacaaacagttccttaagtactcttggcagcctgattcatgcaccgtcccaaggtttgatggggaagcgtttttgaagaaatggagagggaaacgagtgatgttcgtgggtgactcactgagtctaaacatgtgggaatcgttggcatgtatgatacattcgtcggttccaaacactaagaccacttttctcaagcgaaccccactctcctctctcactttccaggaatatgacgtcacactattcctataccgaacaccatacctagtggacatctccaaagaaagtgtcgggcgtgtgcttaaccttggagccattgaagatggagctgatgcttggaaaaatatggacctcctcgtcttcaattcttggcactggtggactcacacaggagtacagtctcaagggtgggattttataagagatgggtcttcactgatgagagacatggaccgccttgatgctttcaacaaaggactcaccacttggggtcaatgggttgatcaaaatgttaatgtttcgcaaacccgagtcttcttccaaggcatttctcccactcactacatgggaagggaatggaacgagccgaggaaaacttgcaacgggcagatgcaaccgttgaccggatcaacatacccaggtggttcacttcctgcagcaagcattgtgtctcgagtattaagcacgatgagaacgcccgtttacttactcgacatcacaactctatctcaactgagaaaagatgctcatccatctacatatggaggcgatggcggaacggattgcagtcactggtgccttcctggcttgccggatacttggaaccagcttctctatgcagctctttcgatgtga

SEQ ID NO: 40 - protein, 385 aa, Arabidopsis thaliana
MGFKLNSLFLLLSLLILTILSGADQALAYVKKPHVSQRNKTALAAVAGRGGGKEMLKGRKQTSGCNLFQGRWVFDASYPFYDSSTCPFIDGEFDCLKFGRPDKQFLKYSWQPDSCTVPRFDGEAFLKKWRGKRVMFVGDSLSLNMWESLACMIHSSVPNTKTTFLKRTPLSSLTFQEYDVTLFLYRTPYLVDISKESVGRVLNLGAIEDGADAWKNMDLLVFNSWHWWTHTGVQSQGWDFIRDGSSLMRDMDRLDAFNKGLTTWGQWVDQNVNVSQTRVFFQGISPTHYMGREWNEPRKTCNGQMQPLTGSTYPGGSLPAASIVSRVLSTMRTPVYLLDITTLSQLRKDAHPSTYGGDGGTDCSHWCLPGLPDTWNQLLYAALSM

SEQ ID NO: 41 - DNA, 1194 bp, Arabidopsis thaliana
  CDS (1)..(1194)
atgttacagtccattcatcttcgtttttcctccacaccatcaccttctaaaagagaatctctcataattccatcggttatttgctcatttcctttcacctcttcttcgttccgtccaaagcaaacccagaaactgaagcgtctggttcaattttgcgctccttacgaggtcggaggtggatacaccgatgaagaattgttcgaaagatacggaactcagcaaaatcaaactaatgtcaaagataaattagatccagctgagtatgaagctttgcttaaaggaggcgaacaagtgacatccgttcttgaagaaatgattaccctcttggaagatatgaagatgaatgaagcatctgagaatgttgctgtagaattggctgcacaaggagttatagggaaaagggtcgatgaaatggaatcagggtttatgatggctcttgattacatgatccaacttgcagacaaagaccaagacgagaagagaaagtctttactagaggtcgtcaaggagactgtcttatctcatctcaccaagaaatgccctcctcatgtccaggtgattggtttactctgtagaaccccgaaaaaggaaagtagacatgagcttctgcgtagggtggctgcaggtggtggggcttttgaaagtgagaacggtactaaacttcatatacccggagcaaatctgaatgacatagctaatcaagctgatgacttgctagagactatggaaacaaggccagctattccggatcgaaaactactagcgaggcttgttttgattagagaggaagcccggaacatgatgggaggaggtatacttgatgaaagaaatgaccgaggtttcactactcttcctgaatcagaggtgaatttcttagccaaattggtagctctgaaacctggaaagactgtgcagcagatgatccagaatgtaatgcaagggaaagatgaaggcgcagataatcttagcaaagaatccgattcttctaccgaaggaagaaaaccaagtggattaaaaggaaggggaagcgttacaggaagaaaaccgttaccagtaagaccaggaatgtttctagaaactgtcacaaaggtactgggaagtatatactcgggtaatgcctccgggataacagcacaacatctagaatgggttcaccaaaaaaccctccaagttcttgaagaaatcgcttattag

SEQ ID NO: 42 - protein, 397 aa, Arabidopsis thaliana
MLQSIHLRFSSTPSPSKRESLIIPSVICSFPFTSSSFRPKQTQKLKRLVQFCAPYEVGGGYTDEELFERYGTQQNQTNVKDKLDPAEYEALLKGGEQVTSVLEEMITLLEDMKMNEASENVAVELAAQGVIGKRVDEMESGFMMALDYMIQLADKDQDEKRKSLLEVVKETVLSHLTKKCPPHVQVIGLLCRTPKKESRHELLRRVAAGGGAFESENGTKLHIPGANLNDIANQADDLLETMETRPAIPDRKLLARLVLIREEARNMMGGGILDERNDRGFTTLPESEVNFLAKLVALKPGKTVQQMIQNVMQGKDEGADNLSKESDSSTEGRKPSGLKGRGSVTGRKPLPVRPGMFLETVTKVLGSIYSGNASGITAQHLEWVHQKTLQVLEEIAY

SEQ ID NO: 43 - DNA, 2451 bp, Arabidopsis thaliana
  CDS (1)..(2451)
atgttgagacaaattccaacctgcaaaggtgggcgctttacacgagttttgcaatctatcggatcagttattagagaattttcagcatctgcaaatgctttgcaagactgttggaaaaatgggaatgaaagtaaagagattgatgatgttcacacacttttcagatattgcactaatttgcaatcagccaaatgtctccacgcacgtctcgttgtctccaaacaaatccaaaatgtttgtatctctgctaaacttgttaatctctattgttaccttggcaatgtcgcattggctaggcacacgtttgatcatatccagaacagagatgtatacgcttggaatttgatgatttctggttatggccgtgcgggaaattcttcagaagtgattaggtgttttagtctctttatgttgagttctggacttacacctgattacaggacttttccttcggttttgaaagcttgtaggactgtgattgatgggaataagattcattgcttggctttgaagtttgggtttatgtgggatgtctatgttgctgcctcgttgattcatttgtactctcgatataaagctgtggggaatgcacgtatactgtttgatgaaatgcctgtaagagatatgggttcttggaatgcaatgatttctgggtattgtcaaagcggaaatgctaaagaggcgttaactctgtctaatgggttgagagctatggattcagttaccgttgtgagtctcctttcggcttgcactgaagccggtgatttcaatagaggagttactattcattcatactcgataaaacatggtctggagtccgaattgtttgtttccaacaagctgattgatttgtatgctgaatttggtaggcttagggattgccagaaggtttttgataggatgtatgtgagggatttgatttcttggaattcgataattaaggcgtacgagctgaatgagcagccgcttagagcgatttcgttgtttcaggagatgcggttgagtagaattcaaccagattgccttactttgattagtttggcttctatactatcacagttaggggacattcgtgcttgtagatctgtgcagggtttcactttgaggaaaggctggttcttggaagatattaccatcgggaatgctgttgtggttatgtatgcaaaactaggccttgtagattcagcaagagcagtgtttaattggcttccgaatacagatgttatttcatggaataccataatctcgggttatgctcaaaacggttttgctagtgaagcaattgaaatgtataacataatggaggaagaaggtgaaatagccgctaaccaagggacttgggtgagtgttttgcccgcttgttcgcaggctggagcgttgcgtcaaggtatgaaactccatggccgtctcttgaagaacggtctttatttggatgtttttgttgttacaagccttgctgatatgtatggaaagtgtggacgactagaggatgccctctctttgttttatcaaatcccaagagtcaactctgtgccatggaacaccctaatagcgtgtcacgggtttcacgggcatggcgagaaagcagtgatgctgttcaaagaaatgctggatgaaggagtgaagccagatcacattacatttgtgactctgttatcggcttgtagccattctgggttagtagacgagggtcaatggtgctttgagatgatgcaaacggattatgggataacaccgagcttgaaacattacggttgtatggtagatatgtatggtcgagctggtcagttagaaaccgcgttgaagtttataaaaagtatgtcgcttcagcctgatgcatccatttggggagctcttctcagcgcttgtagagtccatgggaatgttgatcttggcaagatcgcatctgaacatttgttcgaggttgaacctgagcatgtcgggtatcatgttctattgtcaaacatgtatgctagtgctggaaaatgggaaggagttgatgaaatacgttcaatagcccatggcaaaggtttgaggaagacaccgggttggagctcaatggaagtagacaacaaagtcgaagtcttttacacgggaaaccaaacgcatccaatgtatgaagagatgtacagagaattaacggcattacaagcgaaactgaagatgattgggtatgtaccggaccatagatttgtgttgcaggatgttgaagacgacgagaaagagcatatcctcatgagtcatagtgaaagattagctattgcatttgcacttatcgccactcctgcaaagacaacgattcggatttttaagaatctaagggtatgcggcgattgccatagcgtaaccaagttcatatcgaaaatcactgagagagagatcattgtgagggattcaaaccggtttcatcatttcaagaatggagtctgctcttgcggtgattactggtga

SEQ ID NO: 44 - protein, 816 aa, Arabidopsis thaliana
MLRQIPTCKGGRFTRVLQSIGSVIREFSASANALQDCWKNGNESKEIDDVHTLFRYCTNLQSAKCLHARLVVSKQIQNVCISAKLVNLYCYLGNVALARHTFDHIQNRDVYAWNLMISGYGRAGNSSEVIRCFSLFMLSSGLTPDYRTFPSVLKACRTVIDGNKIHCLALKFGFMWDVYVAASLIHLYSRYKAVGNARILFDEMPVRDMGSWNAMISGYCQSGNAKEALTLSNGLRAMDSVTVVSLLSACTEAGDFNRGVTIHSYSIKHGLESELFVSNKLIDLYAEFGRLRDCQKVFDRMYVRDLISWNSIIKAYELNEQPLRAISLFQEMRLSRIQPDCLTLISLASILSQLGDIRACRSVQGFTLRKGWFLEDITIGNAVVVMYAKLGLVDSARAVFNWLPNTDVISWNTIISGYAQNGFASEAIEMYNIMEEEGEIAANQGTWVSVLPACSQAGALRQGMKLHGRLLKNGLYLDVFVVTSLADMYGKCGRLEDALSLFYQIPRVNSVPWNTLIACHGFHGHGEKAVMLFKEMLDEGVKPDHITFVTLLSACSHSGLVDEGQWCFEMMQTDYGITPSLKHYGCMVDMYGRAGQLETALKFIKSMSLQPDASIWGALLSACRVHGNVDLGKIASEHLFEVEPEHVGYHVLLSNMYASAGKWEGVDEIRSIAHGKGLRKTPGWSSMEVDNKVEVFYTGNQTHPMYEEMYRELTALQAKLKMIGYVPDHRFVLQDVEDDEKEHILMSHSERLAIAFALIATPAKTTIRIFKNLRVCGDCHSVTKFISKITEREIIVRDSNRFHHFKNGVCSCGDYW

SEQ ID NO: 45 - DNA, 3156 bp, Arabidopsis thaliana
  CDS (1)..(3156)
atgggcgaagaatcagaaatcgccatggcttcagcagatcgaaataacctaacccagaaaatcgccaagatcctaaacgagactcgcacatcatacgctactcataaccgtaagctaaaggagctcgccacaatccgttcaaaactatcttcatctgaatccgaatccgagtccgtgtcttcttctattcttcaattctcttccgtgtttctcaaaaccctaacccctcttttcatcgcggctcaacgaagaaccgctgcagcagagcgtgttgttcgattcgtggcggaattcgcttgtcttcgtagtaattcagacggtgactccgattgcgacgagtttctagaggaatttcttaagtttttggttgctgggtctgttgctgccaatagaaacgcgagattcagggcgtgtcagataatttcagagatcatattgaggctgccagatgaagtggaagtagcggatgaactatgggatgatgtaattgattgtatgatgttgcgagttcgggacaaagttcctgttatacgtacatttgctgtgaggtctctttcacgttttgtgaatgatcctgagaatagtgacattcttgatttgctacttgaggtgcttcctttggaacagaatccggaggtacgcaaaacaattgttttatctctgcctccttcaaatgcaaccacccaagcaattattgattgcacgctcgatgttaatgaatcagtacgcaaagcagcttactctgttctggctaataaagttcctcttcagagtctgagcatcaagctaaggaccacaattcttcagagagggctcgctgatcgtgctgtaaatgtttcaacggaatgcttgaagctaatgaaagagcaatggctagctaactattgtgaaggagatcctatcacatttcttaaataccttgacgttgaaacctacgaatctgtggcagaatcggcgttggaagttctgttgagtgaagggttgataatgccctccgatgataaaagcatccagcagtacatattgtcagcagatggtgaaactagagatgaaagcacatgttctgcacccagcattcaacttatggagccagagattgctctttactggaggattatatgcaggaaggtgcaccaaagtgctcaagcaaaaggttctgatgctgctacagcgatgggtgctgaagcagcagtttatgctgctgaagcttcagatgctaatgatttgctggaaagaattcttcctgcaacagtttctgattatgttgatctagttaaagctcatatagaagcaggaccaaatcatcactttgcttcgaggcagctattattgttgggtacgatgcttgatttctctgatgctatgctccacaagactgtgagctcatttgtccaggagctgctccgcagaccttttgagcaagaattagatgaagatgggaacagtattgtgattggagatggtataaaccttggtggtgataaagattgggctgaggcagtgtcgaaattggctaaaaaagtccatgctgcccctggagaatatgaagaagttatacttgttgttgttgaagaagtagcgagaccctgcagggaaaggactgcggatttcctgcagtggatgcacatgctttctctgacaagtcttctcttggaaaatggaaaatctttacattcactgcaagggaaggctattgaaccagaagagatattgcatgctctattgcttccaggggcaaaacacactcacttggatgtgcagaggattgctataaagggccttggtctttttggtttgttagagaagaagcctagcgaagagctagtaaggcagctacgtgcagctttttgcagaagccctcctccaattagtattatggcttgcaaggcactagtggatcttgggatgtggcatagcccaactgaagttgacaaggcaatgggacaagatctcttgtcacaatttgaggatgacagcattgattttgcacccatcgacttatccaatgccgaagaagatatgaactttaaaatgctcgatctcttgtatgctggacttgaaagtgatgactggagagcatccacagagagcagtgagaatgagtcagttaaagcaactgttggggaggggtttgcaaaacttcttcttctaggagagaagtacccaaacttgcctgcatctttctatccgtttgtattgggaaagctaattgctttatatttcagtgaggagtcaaaagaacaactgaggtttaaacagtgtttatccgtcttctttgagcactatgcctccctctcagaaaaacataagggatacgtgtcaaaggcttttgttcctcttgtacgctcaatgtggcctgggattgatggaaacactaaaagttcgtcatatgttgtatcaaatcaacgcaagcgcgcagtccaagtatccagattcatcctacagatgatgcaaaccccgctatacaaaaaagagacaagaggtgagcctgaaagccaggtcaataaatcaccagaagattctatccaacatccactaaactgtacagaagaaggtttggccatacgcatagccatcgagatgctaagcttcaaagaaaagaagactgctcatgagaaggcatatgttgcagcgctgtgcaaaatacttgtgctgctccatctgaaaccatcagaacaaaatgtgacaaagttgttgaaaaagcttttaagcctactcgcagattctgtacgttcagagaaggatcttcttaaagaagtgaaaccggtgcttcaacatctaaaatctttggatgcttgtccaagcgaggagcttacacaagatcaagcaaattccatctttgaaatactcggagttagctacaacttagagatcactgaaactacaacagtgccacagacacctgctccttgctcaacaaaaccagctcggtcaagaagacgagcaaggattgaagaaacatcctctgatgaagaagaagtagcatctcctccaccttctgctcctaatactttgatgacccgatcacaccgtgcaagtaaagctgctgccttggccaaaataatggcaagcaaagtgaaaatgagcaatgtagacgaggatgatgaggaagaagaaggttcttctgatgttacagcagatgattcagatgaataa

SEQ ID NO: 46 - protein, 1051 aa, Arabidopsis thaliana
MGEESEIAMASADRNNLTQKIAKILNETRTSYATHNRKLKELATIRSKLSSSESESESVSSSILQFSSVFLKTLTPLFIAAQRRTAAAERVVRFVAEFACLRSNSDGDSDCDEFLEEFLKFLVAGSVAANRNARFRACQIISEIILRLPDEVEVADELWDDVIDCMMLRVRDKVPVIRTFAVRSLSRFVNDPENSDILDLLLEVLPLEQNPEVRKTIVLSLPPSNATTQAIIDCTLDVNESVRKAAYSVLANKVPLQSLSIKLRTTILQRGLADRAVNVSTECLKLMKEQWLANYCEGDPITFLKYLDVETYESVAESALEVLLSEGLIMPSDDKSIQQYILSADGETRDESTCSAPSIQLMEPEIALYWRIICRKVHQSAQAKGSDAATAMGAEAAVYAAEASDANDLLERILPATVSDYVDLVKAHIEAGPNHHFASRQLLLLGTMLDFSDAMLHKTVSSFVQELLRRPFEQELDEDGNSIVIGDGINLGGDKDWAEAVSKLAKKVHAAPGEYEEVILVVVEEVARPCRERTADFLQWMHMLSLTSLLLENGKSLHSLQGKAIEPEEILHALLLPGAKHTHLDVQRIAIKGLGLFGLLEKKPSEELVRQLRAAFCRSPPPISIMACKALVDLGMWHSPTEVDKAMGQDLLSQFEDDSIDFAPIDLSNAEEDMNFKMLDLLYAGLESDDWRASTESSENESVKATVGEGFAKLLLLGEKYPNLPASFYPFVLGKLIALYFSEESKEQLRFKQCLSVFFEHYASLSEKHKGYVSKAFVPLVRSMWPGIDGNTKSSSYVVSNQRKRAVQVSRFILQMMQTPLYKKETRGEPESQVNKSPEDSIQHPLNCTEEGLAIRIAIEMLSFKEKKTAHEKAYVAALCKILVLLHLKPSEQNVTKLLKKLLSLLADSVRSEKDLLKEVKPVLQHLKSLDACPSEELTQDQANSIFEILGVSYNLEITETTTVPQTPAPCSTKPARSRRRARIEETSSDEEEVASPPPSAPNTLMTRSHRASKAAALAKIMASKVKMSNVDEDDEEEEGSSDVTADDSDE

SEQ ID NO: 47 - DNA, 1695 bp, Arabidopsis thaliana
  CDS (1)..(1695)
atggtgttctctaactctctcctctcttcgaccacttcgcttaccacatgggcaaatcaatcaagttctcagtcaagtctttccccgagatattcgacatggcagtgtgtttgtttacgtaatcagaaacgcaaacctaagctctacttgattcccgctcgccattttttatcgactccaattgattcagtctcctcctcctcaataacggcttcacgttatgctacttctggcgtttctgaagttcaaaggtctacttcgagcaacaatgttactgagatggaagagtttgagatggagttgcaagagttgtttaatgaagtcaaaagtatggtgaagattgggaaggaaagtgacgcaatggaccttcttcgagcgaattatgttgctgtgaaagaagagctggattcgggtttgaaaggtatcgaacaagctgctgttcttgacatcattgctttggggtatatggctgttggagacttgaaacctgtccccgcattgcttgatatgataaacaagattgttgataatttaaaagactccgaacctcttttggattcagtacttatgcatgttggcagtatgtattcggtaatagggaagtttgaaaacgctatactcgtgcatcaaagggctattcgtatattagagaatagatatggtaaatgcaatactttacttgtcacaccattacttggtatggctaagagttttgcttctgatggaaaagccactaaagcgattggtgtttatgagcgtacgttgactatcttagaacggaatagaggttctgagagtgaggatctagtagttccgttattttcactcggtaaacttctgctcaaagaaggtaaagctgctgaagcagaaattccttttaccagcattgtaaatatatacaagaagatatacggagagagagatggaagagttggcatggccatgtgttcccttgctaatgctaagtgctcaaaaggtgatgcaaatgaagctgttgatatctacaggaatgctctacgaataatcaaagactcaaattatatgacaatagacaacagtatcttggaaaacatgaggatagatcttgctgagctgcttcatttcgttggaaggggagatgaaggacgagagttactagaagaatgcttattaataaatgagagattcaaagggaaaaaccatcctagcatggctacacatcttataaaccttgcagcatcttattcgcgttccaagaattatgtggaggcagaacgattgctacgaacttgtttgaacatcatggaggtatcagttggctcagaaggtcagtccataaccttcccaatgctaaatcttgcagtcactctttcccaactgaatcgtgatgaggaagccgagcaaatagccttaaaggttctacgcatccgtgagaaggcatttggcgaagactctctccctgttggagaggcgctggactgtttggtatcgatccaagcgagactaggaagagatgatggagaaatattgggtttgctgaaaagggttatgatgatccaagagaaagagtttggtccttcagctcaagaactcattgttactctccagaagatcattcatttcttggagaaattggagatgaaagatgataaatttaagtttaggagaagattagctttacttagagagagatacaagcagagtcttagctactag

SEQ ID NO: 48 - protein, 564 aa, Arabidopsis thaliana
MVFSNSLLSSTTSLTTWANQSSSQSSLSPRYSTWQCVCLRNQKRKPKLYLIPARHFLSTPIDSVSSSSITASRYATSGVSEVQRSTSSNNVTEMEEFEMELQELFNEVKSMVKIGKESDAMDLLRANYVAVKEELDSGLKGIEQAAVLDIIALGYMAVGDLKPVPALLDMINKIVDNLKDSEPLLDSVLMHVGSMYSVIGKFENAILVHQRAIRILENRYGKCNTLLVTPLLGMAKSFASDGKATKAIGVYERTLTILERNRGSESEDLVVPLFSLGKLLLKEGKAAEAEIPFTSIVNIYKKIYGERDGRVGMAMCSLANAKCSKGDANEAVDIYRNALRIIKDSNYMTIDNSILENMRIDLAELLHFVGRGDEGRELLEECLLINERFKGKNHPSMATHLINLAASYSRSKNYVEAERLLRTCLNIMEVSVGSEGQSITFPMLNLAVTLSQLNRDEEAEQIALKVLRIREKAFGEDSLPVGEALDCLVSIQARLGRDDGEILGLLKRVMMIQEKEFGPSAQELIVTLQKIIHFLEKLEMKDDKFKFRRRLALLRERYKQSLSY

SEQ ID NO: 49 - DNA, 18 bp, artificial sequence
  primer
ngttgwgnatwtsgwgnt

SEQ ID NO: 50 - DNA, 16 bp, artificial sequence
  primer
ngtcgaswganawgaa

SEQ ID NO: 51 - DNA, 16 bp, artificial sequence
  primer
tgwgnagsancasaga

SEQ ID NO: 52 - DNA, 16 bp, artificial sequence
  misc_feature (1)..(16)
agwgnagwancawagg

SEQ ID NO: 53 - DNA, 16 bp, artificial sequence
  misc_feature (1)..(16)
sttgntastnctntgc

SEQ ID NO: 54 - DNA, 15 bp, artificial sequence
  misc_feature (1)..(15)
ntcgastwtsgwgtt

SEQ ID NO: 55 - DNA, 16 bp, artificial sequence
  primer
wgtgnagwancanaga

SEQ ID NO: 56 - DNA, 30 bp, artificial sequence
  primer
actagctctaccgtttccgtttccgtttac

SEQ ID NO: 57 - DNA, 30 bp, artificial sequence
  primer
ttacctcgggttcgaaatcgatcgggataa

SEQ ID NO: 58 - DNA, 33 bp, artificial sequence
  primer
aaaatcggttatacgataacggtcggtacggga

SEQ ID NO: 59 - DNA, 36 bp, artificial sequence
  primer
gggtcttgcggatctgaatatatgttttcatgtgtg

SEQ ID NO: 60 - DNA, 37 bp, artificial sequence
  misc_feature (1)..(37)
taccgaagaaaaataccggttcccgtccgatttcgac

SEQ ID NO: 61 - DNA, 34 bp, artificial sequence
  primer
ggatcgtatcggttttcgattaccgtatttatcc

SEQ ID NO: 62 - DNA, 29 bp, artificial sequence
  primer
attaggcaccccaggctttacactttatg

SEQ ID NO: 63 - DNA, 30 bp, artificial sequence
  primer
gtatgttgtgtggaattgtgagcggataac

SEQ ID NO: 64 - DNA, 30 bp, artificial sequence
  primer
taacaatttcacacaggaaacagctatgac

SEQ ID NO: 65 - DNA, 34 bp, artificial sequence
  primer
tagcatctgaatttcataaccaatctcgatacac

SEQ ID NO: 66 - DNA, 34 bp, artificial sequence
  primer
gcttcctattatatcttcccaaattaccaataca

SEQ ID NO: 67 - DNA, 34 bp, artificial sequence
  primer
gccttttcagaaatggataaatagccttgcttcc

SEQ ID NO: 68 - DNA, 32 bp, artificial sequence
  primer
caaactaggataaattatcgcgcgcggtgtca

SEQ ID NO: 69 - DNA, 32 bp, artificial sequence
  primer
ggtgtcatctatgttactagatcgggaattga

SEQ ID NO: 70 - DNA, 30 bp, artificial sequence
  primer
cgccatggcatatgctagcatgcataattc

SEQ ID NO: 71 - DNA, 35 bp, artificial sequence
  primer
aattggtaattactctttcttttcctccatattga

SEQ ID NO: 72 - DNA, 31 bp, artificial sequence
  primer
atattgaccatcatactcattgctgatccat

SEQ ID NO: 73 - DNA, 28 bp, artificial sequence
  primer
tgatccatgtagatttcccggacatgaa

(19) United States
(12) Patent Application Publication
(10) Pub. No.: US 2004/0191850 A1
(43) Pub. Date: Sep. 30, 2004

(54) Nucleic acid molecules encoding proteins essential for plant growth and development and uses thereof

(75) Inventors: Joshua Zvi Levin, Rockville, MD (US); John Tossberg, Durham, NC (US); Qing Zhou, Raleigh, NC (US); George Aux, Research Triangle Park, NC (US); John McElver, Durham, NC (US); Carla Thomas, Taylors, SC (US); David Andrew Patton, Basel (CH); Catherine Frye, Research Triangle Park, NC (US); Gregory Budziszewski, Durham, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/696,616

(22) Filed: Oct. 29, 2003

Related U.S. Application Data
(60) Provisional application No. 60/423,519, filed on Nov. 4, 2002.

(51) Int. Cl. C12Q 1/48; A01N 63/00

(52) U.S. Cl. 435/15; 504/117

(57) ABSTRACT

Nucleotide sequences are isolated from Arabidopsis thaliana that code for proteins essential for plant growth and development. The essentially of the proteins may be exploited by recombinantly expressing the proteins and using them in screening assays to identify compounds that interact with or inhibit the proteins and are therefore potential herbicides.

This application claims the benefit of U.S. Provisional Application No. 60/423,519 filed Nov. 4, 2002, which is incorporated herein by reference.

### FIELD OF THE INVENTION

The present invention pertains to nucleic acid molecules isolated from Arabidopsis thaliana comprising nucleotide sequences that encode proteins essential for plant growth and development. The invention particularly relates to methods of using these proteins as herbicide targets, based on this essentiality.

### BACKGROUND OF THE INVENTION

The use of herbicides to control undesirable vegetation such as weeds in crop fields has become almost a universal practice. The herbicide market exceeds 15 billion dollars annually. Despite this extensive use, weed control remains a significant and costly problem for farmers.

Effective use of herbicides requires sound management. For instance, the time and method of application and stage of weed plant development are critical to achieving good weed control with herbicides. Because various weed species are resistant to herbicides, the production of effective new herbicides becomes increasingly important. New herbicides can now be discovered using high-throughput screens that implement recombinant DNA technology. Metabolic enzymes found to be essential to plant growth and development can be recombinantly produced through standard molecular biological techniques and utilized as herbicide targets in screens for novel inhibitors of the enzyme activity. More generally, any essential plant protein can be used to screen for inhibitors of its activity. The novel inhibitors discovered through such screens may then be used as herbicides to control undesirable vegetation.

In view of the above, there remain persistent and ongoing problems with unwanted or detrimental vegetation growth (e.g. weeds). Furthermore, as the population continues to grow, there will be increasing food shortages. Therefore, there exists a long felt, yet unfulfilled need, to find new, effective, and economic herbicides.

### SUMMARY OF THE INVENTION

In view of these needs, it is an object of the invention to provide nucleic acid molecules from Arabidopsis thaliana comprising nucleotide sequences that encode proteins essential for plant growth and development. It is another object to provide the essential proteins encoded by these essential nucleotide sequences for assay development to identify inhibitory compounds with herbicidal activity. It is still another object of the present invention to provide an effective and beneficial method for identifying new or improved herbicides using the essential proteins of the invention.

In furtherance of these and other objects, the present invention provides nucleic acid molecules isolated from Arabidopsis thaliana comprising nucleotide sequences that encode proteins essential for plant viability. Genetic results show that when any of the nucleotide sequences of the invention are mutated in Arabidopsis thaliana, the resulting phenotype is embryo or seedling lethal in the homozygous state. In particular, by using Acids transposon or T-DNA-mediated mutagenesis, the inventors of the present invention are the first to demonstrate that the activity of each protein of the present invention is essential for plant growth in Arabidopsis thaliana.

This knowledge is exploited to provide novel herbicide modes of action. The critical role in plant growth of the proteins encoded by each of the nucleotide sequences of the invention implies that chemicals that inhibit the function of any one of these proteins in plants are likely to have detrimental effects on plants and are potentially good herbicide candidates. Thus, the proteins encoded by the essential nucleotide sequences provide the bases for assays designed to easily and rapidly identify novel herbicides.

The present invention therefore provides methods of using a purified protein encoded by any one of the nucleotide sequences described below to identify inhibitors thereof, which can then be used as herbicides to suppress the growth of undesirable vegetation, e.g. in fields where crops are grown, particularly agronomically important crops such as maize and other cereal crops such as wheat, oats, rye, sorghum, rice, barley, millet, turf and forage grasses, and the like, as well as cotton, sugar cane, sugar beet, oilseed rape, and soybeans.

Disclosed herein are nucleic acid molecules isolated from Arabidopsis thaliana. In one embodiment, the present invention provides an isolated nucleic acid molecule comprising a nucleotide sequence, the complement of which hybridizes under stringent conditions to a sequence selected from the group consisting of the odd numbered SEQ ID NOs: 1-47. In another embodiment, the present invention provides an isolated nucleic acid molecule comprising a nucleotide sequence that encodes a protein comprising an amino acid sequence having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-48.

The present invention also provides a chimeric construct comprising a promoter operatively linked to a nucleic acid molecule according to the present invention, wherein the promoter is preferably functional in a eukaryote, wherein the promoter is preferably heterologous to the nucleic acid molecule. The present invention further provides a recombinant vector comprising a chimeric construct according to the present invention, wherein said vector is capable of being stably transformed into a host cell. The present invention still further provides a host cell comprising a nucleic acid molecule according to the present invention, wherein said nucleic acid molecule is preferably expressible in the cell. The host cell is preferably selected from the group consisting of a plant cell, a yeast cell, an insect cell, and a prokaryotic cell. The present invention additionally provides a plant or seed comprising a plant cell according to the present invention.

The present invention also provides proteins essential for plant growth in Arabidopsis thaliana. In one embodiment, the present invention provides an isolated protein comprising an amino acid sequence having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-48. In accordance with another embodiment, the present invention also relates to the recombinant production of proteins of the invention and methods of using the proteins of the invention in assays for identifying compounds that interact with the protein.

According to another aspect, the present invention provides a method of identifying a herbicidal compound, comprising: (a) combining a polypeptide comprising an amino acid sequence at least 90% identical to an amino acid sequence selected from the group consisting of the even numbered SEQ ID NOs:2-48 with a compound to be tested for the ability to bind to said polypeptide, under conditions conducive to binding; (b) selecting a compound identified in (a) that binds to said polypeptide; (c) applying a compound selected in (b) to a plant to test for herbicidal activity; and (d) selecting a compound identified in (c) that has herbicidal activity. Preferably, the polypeptide comprises an amino acid sequence at least 95% identical to an amino acid sequence selected from the group consisting of the even numbered SEQ ID NOs:2-48. More preferably, the polypeptide comprises an amino acid sequence at least 99% identical to an amino acid sequence selected from the group consisting of the even numbered SEQ ID NOs:2-48. Most preferably, the polypeptide comprises an amino acid sequence selected from the group consisting of the even numbered SEQ ID NOs:2-48. The present invention also provides a method for killing or inhibiting the growth or viability of a plant, comprising applying to the plant a herbicidal compound identified according to this method.

According to yet another aspect, the present invention provides a method of identifying a herbicidal compound, comprising: (a) combining a polypeptide comprising an amino acid sequence at least 90% identical to an amino acid sequence selected from the group consisting of the even numbered SEQ ID NOs:2-48 with a compound to be tested for the ability to inhibit the activity of said polypeptide, under conditions conducive to inhibition; (b) selecting a compound identified in (a) that inhibits the activity of said polypeptide; (c) applying a compound selected in (b) to a plant to test for herbicidal activity; and (d) selecting a compound identified in (c) that has herbicidal activity. Preferably, the polypeptide comprises an amino acid sequence at least 95% identical to an amino acid sequence selected from the group consisting of the even numbered SEQ ID NOs:2-48. More preferably, the polypeptide comprises an amino acid sequence at least 99% identical to an amino acid sequence selected from the group consisting of the even numbered SEQ ID NOs:2-48. Most preferably, the polypeptide comprises an amino acid sequence selected from the group consisting of the even numbered SEQ ID NOs:2-48. The present invention also provides a method for killing or inhibiting the growth or viability of a plant, comprising applying to the plant a herbicidal compound identified according to this method.

The present invention still further provides a method for killing or inhibiting the growth or viability of a plant, comprising inhibiting expression in said plant of a protein having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-48.

Other objects and advantages of the present invention will become apparent to those skilled in the art and from a study of the following description of the invention and non-limiting examples. The entire contents of all publications mentioned herein are hereby incorporated by reference.

### BRIEF DESCRIPTION OF THE SEQUENCES IN THE SEQUENCE LISTING

Odd numbered SEQ ID NOs: 1-47 are nucleotide sequences isolated from Arabidopsis thaliana that are more fully described in Table 5 below.

Even numbered SEQ ID NOs:2-48 are protein sequences encoded by the immediately preceding nucleotide sequence, e.g., SEQ ID NO:2 is the protein encoded by the nucleotide sequence of SEQ ID NO: 1, SEQ ID NO:4 is the protein encoded by the nucleotide sequence of SEQ ID NO:3, etc.

SEQ ID NOs:49-73 are PCR primers.

### DEFINITIONS

For clarity, certain terms used in the specification are defined and presented as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Essential: an “essential” Arabidopsis thaliana nucleotide sequence is a nucleotide sequence encoding a protein such as e.g. a biosynthetic enzyme, receptor, signal transduction protein, structural gene product, or transport protein that is essential to the growth or survival of the plant.

Expression Cassette: “Expression cassette” as used herein means a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue or organ or stage of development.

Gene: the term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

Hybridization: The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

Inhibitor: a chemical substance that inactivates the enzymatic activity of a protein such as a biosynthetic enzyme, receptor, signal transduction protein, structural gene product, or transport protein. The term “herbicide” (or “herbicidal compound”) is used herein to define an inhibitor applied to a plant at any stage of development, whereby the herbicide inhibits the growth of the plant or kills the plant.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

Isogenic: plants that are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein from which the transit peptide, signal peptide, and/or propeptide portions have been removed.

Minimal Promoter: the smallest piece of a promoter, such as a TATA element, that can support any transcription. A minimal promoter typically has greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed plant cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene.

“ORF” means open reading frame.

Percent identity: the phrases “percent identical” or “percent identical,” in the context of two nucleic acid or protein sequences, refers to two or more sequences or subsequences that have for example 60%, preferably 70%, more preferably 80%, still more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the percent identity exists over a region of the sequences that is at least about 50 residues in length, more preferably over a region of at least about 100 residues, and most preferably the percent identity exists over at least about 150 residues. In an especially preferred embodiment, the percent identity exists over the entire length of the coding regions.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Pre-protein: protein that is normally targeted to a cellular organelle, such as a chloroplast, and still comprises its native transit peptide.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Specific Binding/Immunological Cross-Reactivity: An indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions. The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.×SSC, 0.1% SDS at 50° C, more preferably in 7% sodium dodecyl sulfate (SDS), 0.5 M NaPO₄, 1 mM EDTA at 50° C. with washing in 0.×SSC, 0.1% SDS at 65° C.

A “subsequence” refers to a sequence of nucleic acids or amino acids that comprise a part of a longer sequence of nucleic acids or amino acids (e.g., protein) respectively.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Transformation: a process for introducing heterologous DNA into a plant cell, plant tissue, or plant. Transformed plant cells, plant tissue, or plants are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of a plant. Plants are assayed for their homozygous performance of plant development, indicating which proteins are essential for plant growth.

### DETAILED DESCRIPTION OF THE INVENTION

I. Identification of Essential Arabidopsis thaliana Nucleotide Sequences and Encoded Proteins Using Acids Transposon or T-DNA-Mediated Mutagenesis

As shown in the examples below, the essentiality of the nucleotide sequences described herein for normal plant growth and development, have been demonstrated for the first time in Arabidopsis using Ac/Ds transposon or T-DNA-mediated mutagenesis. Having established the essentiality of the function of the encoded proteins in Arabidopsis thaliana and having identified the nucleotide sequences encoding these essential proteins, the inventors thereby provide an important and sought after tool for new herbicide development.

Arabidopsis insertional mutant lines segregating for seedling lethal mutations are identified as a first step in the identification of essential proteins. Starting with T2 seeds collected from single Ti plants containing T-DNA insertions in their genomes, those lines segregating homozygous seedling lethal seedlings are identified. Ds transposon insertion lines are produced as described in Sundaresan et al. (1995) (Genes and Dev., 9:1797-1810), incorporated herein by reference. Starting with F3 or F4 seeds collected from single F2 or F3 kanamycin-resistant plants containing Ds insertions in their genomes (see FIG. 3 of Sundaresan et al. (1995) (Genes and Dev., 9:1797-1810), those lines segregating homozygous seedling lethal seedlings are identified. These lines are found by placing seeds onto minimal plant growth media, which contains the fungicides benomyl and maxim, and screening for inviable seedlings after 7 and 14 days in the light at room temperature. Inviable phenotypes include altered pigmentation or altered morphology. These phenotypes are observed either on plates directly or in soil following transplantation of seedlings.

Essential genes are also identified through the isolation of lethal mutants blocked in early development. Examples of lethal mutants include those blocked in the formation of the male or female gametes or embryo. Gametophytic mutants are found by examining T1 insertion lines for the presence of 50% aborted pollen grains or ovules. Embryo defective mutants produce 25% defective seeds following self-pollination of T1 plants (see Errampalli et al. 1991, Plant Cell 3:149-157; Castle et al. 1993, Mol Gen Genet 241:504-514).

When a line is identified as segregating a seedling lethal or an embryo defective phenotype, it is determined if the resistance marker in the Ds transposon or T-DNA insertion co-segregates with the lethality (Errampalli et al. (1991) The Plant Cell, 3:149-157). Cosegregation analysis is done by placing the seeds on media containing the selective agent and scoring the seedlings for resistance or sensitivity to the agent. Examples of selective agents used are kanamycin, hygromycin, or phosphinothricin. About 35 resistant seedlings are transplanted to soil and their progeny are examined for the segregation of the seedling lethal. In the case in which the Ds transposon or T-DNA insertion disrupts an essential gene, there is co-segregation of the resistance phenotype and the seedling lethal or embryo defective phenotype in every plant. Therefore, in such a case, all resistant plants segregate a seedling lethal or embryo defective phenotype in the next generation; this result indicates that each of the resistant plants is heterozygous for the mutation and hemizygous for the T-DNA insert causing the mutation.

For the Arabidopsis lines showing co-segregation of the transposon-encoded or T-DNA-encoded resistance marker and the lethal phenotype, PCR-based molecular approaches such as, TAIL-PCR (Liu et al. (1995) Plant J., 8:457-463; Liu and Whittier (1995), Genomics, 25:674-681), TAIL2k, vectorette PCR (Riley et al. (1990) Nucleic Acids Research, 18: 2887-2890)), or the GenomeWalker™ kit (CLONTECH Laboratories, Inc., Palo Alto, Calif.), may be used to directly amplify the plant DNA fragments flanking the transposon or T-DNA. Each of these techniques utilizes the known sequence of the transposon or T-DNA, and can be used to recover small (less than 5 kb) fragments directly adjacent to the insertion. PCR products are isolated and their DNA sequence is determined.

Alternatively, plasmid rescue may be used to isolate the plant DNA/T-DNA border fragments. Southern blot analysis may be performed as an initial step in the characterization of the molecular nature of each insertion. Southern blots are done with genomic DNA isolated from heterozygotes and using probes capable of hybridizing with the T-DNA vector DNA. Using the results of the Southern analysis, appropriate restriction enzymes are chosen to perform plasmid rescue in order to molecularly clone Arabidopsis thaliana genomic DNA flanking one or both sides of the T-DNA insertion. Plasmids obtained in this manner are analyzed by restriction enzyme digestion to sort the plasmids into classes based on their digestion pattern. For each class of plasmid clone, the DNA sequence is determined.

The resulting sequences, obtained by any of the above outlined approaches, are analyzed for the presence of non-Ds transposon and non-T-DNA vector sequences, as appropriate. When such sequences are found, they are used to search DNA and protein databases using the BLAST and BLAST2 programs (Altschul et al. (1990) J Mol. Biol. 215: 403-410; Altschul et al. (1997) Nucleic Acid Res. 25:3389-3402, both incorporated herein by reference). Additional genomic and cDNA sequences for each gene are identified by standard molecular biology procedures.

II. Recombinant Production of Essential Proteins and Uses Thereof

For recombinant production of a protein of the invention in a host organism, a nucleotide sequence encoding the protein is inserted into an expression cassette designed for the chosen host and introduced into the host where it is recombinantly produced. The choice of the specific regulatory sequences such as promoter, signal sequence, 5′ and 3′ untranslated sequence, and enhancer appropriate for the chosen host is within the level of the skill of the routineer in the art. The resultant molecule, containing the individual elements linking in the proper reading frame, is inserted into a vector capable of being transformed into the host cell. Suitable expression vectors and methods for recombinant production of proteins are well known for host organisms such as E. coli, yeast, and insect cells (see, e.g., Lucknow and Summers, Bio/Technol. 6:47 (1988)). Additional suitable expression vectors are baculovirus expression vectors, e.g., those derived from the genome of Autographica californica nuclear polyhedrosis virus (AcMNPV). A preferred baculovirus/insect system is PVL1392(3) used to transfect Spodoptera frugiperda SF9 cells (ATCC) in the presence of linear Autographica californica baculovirus DNA (Phramingen, San Diego, Calif.). The resulting virus is used to infect HighFive Tricoplusia ni cells (Invitrogen, La Jolla, Calif.).

Recombinantly produced proteins are isolated and purified using a variety of standard techniques. The actual techniques used vary depending upon the host organism used, whether the protein is designed for secretion, and other such factors. Such techniques are well known to the skilled artisan (see, e.g. chapter 16 of Ausubel, F. et al., “Current Protocols in Molecular Biology”, pub. by John Wiley & Sons, Inc. (1994).

III. Assays for Characterizing the Essential Proteins

The recombinantly produced proteins described herein are useful for a variety of purposes. For example, they can be used in in vitro assays to screen known herbicidal chemicals whose target has not been identified to determine if they inhibit protein activity. Such in vitro assays may also be used as more general screens to identify chemicals that inhibit such protein activity and that are therefore novel herbicide candidates. Recombinantly produced proteins may also be used to elucidate the complex structure of these molecules and to further characterize their association with known inhibitors in order to rationally design new inhibitory herbicides. Alternatively, the recombinant protein can be used to isolate antibodies or peptides that modulate the activity and are useful in transgenic solutions.

IV. In vitro Inhibitor Assay: Discovery of Small Molecule Ligands That Interact with Essential Proteins of Unknown Biochemical Function

Once a protein has been identified as a potential herbicide target based on its essentiality for normal plant growth and viability, a next step is to develop an assay that allows screening large number of chemicals to determine which ones interact with the protein. Although it is straightforward to develop assays for proteins of known function, developing assays with proteins of unknown functions can be more difficult.

To address this issue, novel technologies are used that can detect interactions between a protein and a compound without knowing the biological function of the protein. A short description of three methods is presented, including fluorescence correlation spectroscopy, surface-enhanced laser desorption/ionization, and biacore technologies.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-11757). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides a mean to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable to pipette the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microlitre cell with the immobilized protein. Binding is detected by surface plasmon resonance (SPR) by recording laser light refracting from the surface. In general, the refractive index change for a given change of mass concentration at the surface layer, is practically the same for all proteins and peptides, allowing a single method to be applicable for any protein (Liedberg et al. (1983) Sensors Actuators 4: 299-304; Malmquist (1993) Nature, 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipette the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

Another assay for small molecule ligands that interact with a polypeptide is an inhibitor assay. For example, such an inhibitor assay useful for identifying inhibitors of the products of essential plant nucleic acid sequences, such as the essential Arabidopsis proteins described herein, comprises the steps of:

a) reacting an essential Arabidopsis protein described herein and a substrate thereof in the presence of a suspected inhibitor of the protein's function;

b) comparing the rate of enzymatic activity of the protein in the presence of the suspected inhibitor to the rate of enzymatic activity under the same conditions in the absence of the suspected inhibitor; and

c) determining whether the suspected inhibitor inhibits the essential Arabidopsis protein.

For example, the inhibitory effect on the activity of a hereindescribed essential Arabidopsis protein, may be determined by a reduction or complete inhibition of protein activity in the assay. Such a determination may be made by comparing, in the presence and absence of the candidate inhibitor, the amount of substrate used or intermediate or product made during the reaction.

V. Production of Peptides

Phage particles displaying diverse peptide libraries permits rapid library construction, affinity selection, amplification and selection of ligands directed against an essential protein (H. B. Lowman, Annu. Rev. Biophys. Biomol. Struct. 26, 401-424 (1997)). Structural analysis of these selectants can provide new information about ligand-target molecule interactions and then in the process also provide a novel molecule that can enable the development of new herbicides based upon these peptides as leads.

VI. In Vivo Inhibitor Assay

In one embodiment, a suspected herbicide, for example identified by in vitro screening, is applied to plants at various concentrations. The suspected herbicide is preferably sprayed on the plants. After application of the suspected herbicide, its effect on the plants, for example death or suppression of growth is recorded.

In another embodiment, an in vivo screening assay for inhibitors of the activity of a hereindescribed essential protein uses transgenic plants, plant tissue, plant seeds or plant cells capable of overexpressing a nucleotide sequence disclosed herein that encodes an essential protein, wherein the essential protein is enzymatically active in the transgenic plants, plant tissue, plant seeds or plant cells. A chemical is then applied to the transgenic plants, plant tissue, plant seeds or plant cells and to the isogenic non-transgenic plants, plant tissue, plant seeds or plant cells, and the growth or viability of the transgenic and non-transformed plants, plant tissue, plant seeds or plant cells are determined after application of the chemical and compared. Compounds capable of inhibiting the growth of the non-transgenic plants, but not affecting the growth of the transgenic plants are selected as specific inhibitors of the essential protein's activity.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998). These references describe the standard techniques used for all steps in tagging and cloning genes from Ac/Ds transposon or T-DNA mutagenized populations of Arabidopsis: plant infection and transformation; screening for the identification of seedling mutants; and cosegregation analysis. Ds transposon insertion lines produced as described in Sundaresan et al. (1995) Genes and Dev., 9:1797-1810) are used in these experiments. T-DNA lines are generated using vacuum infiltration or floral dip methods (Bechtold et al. (1993) C. R. Acad. Sci. Paris, 316:1194-1199; Clough and Bent (1998) Plant J., 16:735-743; Desfeux et al. (2000) Plant Physiol., 123:895-904).

### Example 1

### Identification of Arabidopsis Mutants with Lethal Phenotypes

Essential genes are identified through the isolation of lethal mutants blocked in early development. Examples of lethal mutants include those blocked in the formation of the male or female gametes, embryo, or resulting seedling. Gametophytic mutants are found by examining insertion lines for the presence of 50% aborted pollen grains or ovules. Embryo defective lethal mutants usually produce 25% defective seeds following self-pollination of plants heterozygous for an insertion (see Errampalli et al. 1991, Plant Cell 3:149-157; Castle et al. 1993, Mol Gen Genet 241:504-514). Seedling lethal mutants usually segregate 25% seedlings that exhibit a lethal phenotype.

### Example 2

### Cosegregation Analysis for Lines with Lethal Phenotypes

The linkage of the mutation to the Ds or T-DNA insertion is established after identifying a transformed line segregating for a lethal phenotype of interest. A line segregating with a single functional insert will segregate for resistance in the ratio of about 2:1 (resistant: sensitive) to the selectable marker. In the case of an embryo defective mutant, one-quarter of the progeny of a plant heterozygous for an insertion will fail to germinate due to embryo lethality, resulting in a reduction of the normal 3:1 ratio to 2:1. In the case of a seedling lethal mutant, the seedlings with a mutant phenotype are excluded in the calculation of this ratio. Each of the resistant progeny is therefore heterozygous for the mutation if the Ds or T-DNA insertion is causing the mutant phenotype. To establish cosegregation of the insertion and the mutant phenotype, about 30 resistant progeny are transplanted to soil and each plant is shown to segregate the 25% progeny with a lethal phenotype by the appropriate screening of embryo or seedlings. When all resistant plants segregate the lethal phenotype, there is cosegregation of the insertion and the lethal mutation and the line is designated as “tagged.”

### Example 3

### T-DNA Border Isolation by Plasmid Rescue

The plasmid rescue technique is used to molecularly clone Arabidopsis flanking DNA from one or both sides of the T-DNA insertion(s). Arabidopsis genomic DNA is isolated as described by Reiter et al. in Methods in Arabidopsis Research, World Scientific Press (1992). Genomic DNA is digested with a restriction endonuclease and ligated overnight. After ligation, the DNA is transformed into competent E. coli strain XL-1 Blue, DH10B, DH5 alpha, or the like, and colonies are selected on semi-solid medium containing ampicillin. Resistant colonies are picked into liquid medium with ampicillin and grown overnight. Plasmid DNA is isolated and digested with the rescue enzyme and analyzed on agarose gels containing ethidium bromide for visualization. Plasmids that represent different size classes are sequenced using primers that flank the plant DNA portion of the rescue element and the sequence is analyzed to determine what portion is plant DNA and what gene has been disrupted. The plasmid rescue is validated via PCR of template genomic DNA from a heterozygote for the insertion mutation. The experiment uses a primer anchored in the predicted flanking sequence and a primer in the T-DNA insertion. Finding a PCR product of the appropriate size, based on the sequence of the plasmid rescue clone confirms a valid rescue. Alternatively, Southern blot analysis with a probe that detects the relevant region of Arabidopsis DNA in genomic DNA from a heterozygote for the insertion mutation can be used to confirm the plasmid rescue results.

### Example 4

### Transposon or T-DNA Border Isolation by TAIL-PCR

Arabidopsis genomic DNA is isolated according to Reiter et al. in Methods in Arabidopsis Research, World Scientific Press (1992) or using the Nucleon PhytoPure™ Plant DNA isolation kit (Amersham International plc, Buckinghamshire, England) or the Puregene DNA isolation kit (Gentra Systems, Minneapolis, Minn.). Fragments of genomic DNA flanking the borders of the transposon or T-DNA are isolated using the TAIL-PCR technique (Liu et al. (1995) Plant J., 8:457-463; Liu and Whittier (1995), Genomics, 25:674-681). Three sets of 12 TAIL-PCR reactions, referred to as the primary, secondary and tertiary reactions, are performed. In each reaction, one arbitrary degenerate primer and one transposon-specific or T-DNA-specific primer are used. The arbitrary degenerate primer is chosen from among seven primers, LWAD 1, CA50, CA51, CA52, CA53, CA54, and CA55 (Table 1), which are used to prime the genomic DNA flanking the insertion. Alternatively, less than 12 TAIL-PCR reactions are done using fewer arbitrary degenerate primers. These degenerate primers are used in combination with two sets of three, nested, transposon-specific primers (Table 2) or T-DNA-specific primers (Table 3). The transposon-specific primers are homologous to regions of the Ds elements that lie at the outermost ends of the transposons, DS5 at the 5′ end (primers 5A, 5B, and 5C) and DS3 at the 3′ end (primers 3A, 3B, and 3C). The T-DNA-specific primers are homologous to regions of the T-DNA that lie in the borders of the T-DNAs. For the pCSA104 and pDAP101 T-DNAs, right borders are recovered with CA66 (primary primer), CA67 (secondary primer), and CA68 (tertiary primer) and left borders are recovered with JM33 (tertiary primer); JM34 (secondary primer); and JM35 (primary primer). For the pCSA110 T-DNA, right borders are recovered with QRB1 (primary primer), QRB2 (secondary primer), and QRB3 (tertiary primer) and left borders are recovered with JM33 (tertiary primer); JM34 (secondary primer); and JM35 (primary primer). For the pPCVICEn4HPT (Hayashi et al. (1992), Science, 258:1350-1353) and pSKI015 (Weigel et al. (2000) Plant Physiol. 122:1003-1014) T-DNAs, left borders are recovered with SKI1 (primary primer), SKI2 (secondary primer), and SKI3 (tertiary primer). When the degenerate and nested primer pairs are used in a series of low and high-stringency PCR amplifications, as described in the TAIL-PCR protocol (Liu and Whittier (1995), Genomics, 25:674-681), DNA fragments are produced that correspond to the genomic DNA that is directly adjacent to the transposon or T-DNA insertion. The nucleic acid sequences of the PCR products from the tertiary TAIL-PCR reactions are then determined by standard molecular biology techniques. The resulting sequences are analyzed for the presence of non-Ds transposon or non-T-DNA vector sequence.

To confirm the integrity of the resultant products, PCR primers specific to the flanking genomic region are designed and used in conjunction with the tertiary nested primer in a PCR reaction, to confirm the transposon or T-DNA insertion point within the genomic DNA. Finding a PCR product of the appropriate size, based on the sequence of the TAIL-PCR clone confirms a valid rescue.

TABLE 1

Arbitrary Degenerate Primers

| | | | |
|---|---|---|---|
| SEQ ID NO: | Primer | Degen. | Primer Sequence |

| | | | |
|---|---|---|---|
| 49 | LWAD1 | 1026 | ngt tgw gna twt sgw gnt |
| 50 | CA50 | 128 | ngt cga swg ana wga a |
| 51 | CA51 | 128 | tgw gna gsa nca sag a |
| 52 | CA52 | 128 | agw gna gwa nca wag g |
| 53 | CA53 | 256 | stt gnt ast nct ntg c |
| 54 | CA54 | 64 | ntc gas twt sgw gtt |
| 55 | CA55 | 256 | wgt gna gwa nca nag a |

TABLE 2

Nested Primers For Ds Lines

| SEQ ID | | |
|---|---|---|
| NO: | Primer | Primer Sequence |
| 56 | 5A | actagctctaccgtttccgtttccgtttac |
| 57 | 5B | ttacctcgggttcgaaatcgatcgggataa |
| 58 | 5C | aaaatcggttatacgataacggtcggtacggga |
| 59 | 3A | gggtcttgcggatctgaatatatgttttcatgtgtg |
| 60 | 3B | taccgaagaaaaataccggttcccgtccgatttcgac |
| 61 | 3C | ggatcgtatcggttttcgattaccgtatttatcc |

TABLE 3

Nested Primers For T-DNA Lines

| | | |
|---|---|---|
| SEQ ID NO: | Primer | Primer Sequence |

| | | |
|---|---|---|
| 62 | CA66 | att agg cac ccc agg ctt tac act tta tg |
| 63 | CA67 | gta tgt tgt gtg gaa ttg tga gcg gat aac |
| 64 | CA68 | taa caa ttt cac aca gga aac agc tat gac |
| 65 | JM33 | tag cat ctg aat ttc ata acc aat ctc gat aca c |
| 66 | JM34 | gct tcc tat tat atc ttc cca aat tac caa tac a |
| 67 | JM35 | gcc ttt tca gaa atg gat aaa tag cct tgc ttc c |
| 68 | QRB1 | caa act agg ata aat tat cgc gcg cgg tgt ca |
| 69 | QRB2 | ggt gtc atc tat gtt act aga tcg gga att ga |
| 70 | QRB3 | cgc cat ggc ata tgc tag cat gca taa ttc |
| 71 | SKI1 | aat tgg taa tta ctc ttt ctt ttc ctc cat att ga |
| 72 | SKI2 | ata ttg acc atc ata ctc att gct gat cca t |
| 73 | SKI3 | tga tcc atg tag att tcc cgg aca tga a |

### Example 5

### Transposon or T-DNA Border Isolation by TAIL2k PCR

Arabidopsis genomic DNA is isolated according to Reiter et al. in Methods in Arabidopsis Research, World Scientific Press (1992) or using the Nucleon PhytoPure™ Plant DNA isolation kit (Amersham International plc, Buckinghamshire, England) or the Puregene DNA isolation kit (Gentra Systems, Minneapolis, Minn.). Fragments of genomic DNA flanking the borders of the transposon or T-DNA are isolated using the TAIL2k PCR technique. Two sets of 12 TAIL-PCR reactions, referred to as the primary and secondary reactions, are performed. In each reaction, one arbitrary degenerate primer and one transposon-specific or T-DNA-specific primer are used. The arbitrary degenerate primer is selected from among six primers; CA50, CA51, CA52, CA53, CA54, and CA55 (Table 1), which are used to prime the genomic DNA flanking the insertion. Alternatively, less than 12 TAIL-PCR reactions are done using fewer arbitrary degenerate primers. These degenerate primers are used in combination with two sets of two, nested, transposon-specific primers (Table 2) or T-DNA-specific primers (Table 3). The transposon-specific primers are homologous to regions of the Ds elements that lie at the outermost ends of the transposons, DS5 at the 5′ end (primers 5A, 5B, and 5C) and DS3 at the 3′ end (primers 3A, 3B, and 3C). The T-DNA-specific primers are homologous to regions of the T-DNA that lie in the borders of the T-DNAs. For the pCSA104 and pDAP101 T-DNAs, right borders are recovered with CA66 (primary primer), CA67 (secondary primer), and CA68 (sequencing primer) and left borders are recovered with JM33 (sequencing primer), JM34 (secondary primer), and JM35 (primary primer). Primers CA66, CA67, and CA68 are also known as RB1, RB2, and RB3, respectively. Primers JM35, JM34, and JM33 are also known as LB1, LB2, and LB3, respectively. For the pCSA110 T-DNA, right borders are recovered with QRB1 (primary primer), QRB2 (secondary primer), and QRB3 (sequencing primer) and left borders are recovered with JM33 (sequencing primer); JM34 (secondary primer); and JM35 (primary primer). For the pPCVICEn4HPT (Hayashi et al. (1992), Science, 258:1350-1353) and pSKI1015 (Weigel et al. (2000) Plant Physiol. 122:1003-1014) T-DNAs, left borders are recovered with SKI1 (primary primer), SK12 (secondary primer), and SKI3 (sequencing primer). When the degenerate and nested primer pairs are used in a series of low and high-stringency PCR amplifications, as described in the TAIL-PCR protocol (Liu and Whittier (1995), Genomics, 25:674-681), DNA fragments are produced that correspond to the genomic DNA that is directly adjacent to the transposon or T-DNA insertion. TAIL2k-PCR differs from the original TAIL-PCR protocol by the elimination of the tertiary PCR and modification of the secondary PCR. The cycling conditions used in the secondary reaction are modified to include 5 high annealing temperature cycles (64 degrees C.) at the beginning, three additional so-called super cycles, and five additional low annealing temperature cycles (44 degrees C.) at the end of the reaction. The melting and extension times are the same as all other TAIL-PCR reactions. Additionally, the reaction volume is increased to 40 microliters. The nucleic acid sequences of the PCR products from the secondary TAIL2k-PCR reactions are then determined by standard molecular biology techniques. The resulting sequences are analyzed for the presence of non-Ds transposon or non-T-DNA vector sequence.

To confirm the integrity of the resultant products, PCR primers specific to the flanking genomic region are designed and used in conjunction with the tertiary nested primer in a PCR reaction, to confirm the transposon or T-DNA insertion point within the genomic DNA. Finding a PCR product of the appropriate size, based on the sequence of the TAIL2k-PCR sequencing result confirms a valid rescue.

### Example 6

### Identification of Both Borders of a T-DNA or Ds Insertion

If the results of border rescue provide information on only one of the two borders for an insertion in a given line, additional experiments are performed to identify the second border. These experiments are necessary to show that a single gene has been disrupted in a given line. In some cases, an insertion can affect more than a single gene due to a chromosomal deletion or rearrangement. In those cases, additional experiments are required to identify which of the affected genes is responsible for the lethal phenotype.

When both borders of an insertion are not recovered, primers are designed to isolate a PCR product that will provide information on the location of the missing border. Three primers are chosen in Arabidopsis genomic DNA on the opposite side of the insertion about one, two, and five kb away from the insertion point; the primers point towards the expected second border. Long PCR conditions (Advantage 2, Clontech) are then employed following the manufacturer's directions to amplify the relevant region from genomic DNA isolated from a heterozygote for the lethal mutation. PCR reactions are performed using appropriate pairs of genomic and T-DNA or Ds border primers. Finding a PCR product of the appropriate size, based on the sequence of the TAIL-PCR clone confirms a valid rescue of the second border. In some cases, the PCR product is directly sequenced to determine the exact insertion point.

If the second border is not recovered with this method, an additional set of PCR reactions are preformed. In these experiments, the genomic primers are paired with a series of internal T-DNA or Ds primers designed at about one kb intervals in both orientations across the entire T-DNA or Ds vector sequence. Finding a PCR product of the appropriate size, based on the sequence of the TAIL-PCR clone confirms a valid rescue of the second border. In some cases, the PCR product is directly sequenced to determine the exact insertion point. Any borders recovered with this approach are classified as abnormal because they lack the ends of the Ds transposon or the expected 24 bp T-DNA imperfect repeat characteristic of right and left borders.

### Example 7

### Identification of Insertion Points for Lines with Lethal Phenotypes

For each line with a lethal phenotype, the sequences of the borders of the insertion are determined and the insertion points in the Arabidopsis genome are deduced. For Ds insertion lines, PCR products are obtained from the Ds3 and Ds5 borders. For T-DNA lines, PCR products or plasmid rescue clones are obtained from left (LB), right (RB), or abnormal (AB) borders. These sequences are used in BLASTn searches against nucleotide databases (Altschul et al. (1990) J Mol. Biol. 215:403-410; Altschul et al. (1997) Nucleic Acids Res. 25:3389-3402). The results are summarized in Table 4. Ds line names begin with ET or GT; T-DNA line names are numbers. The insertion point (Insert Pt.) and the direction of the flanking sequence (Dir.) either up (U) or down (D) in the genome section is noted. Often, small deletions or duplications of genomic DNA accompany the insertion of a T-DNA or Ds transposon.

The gene that has been inactivated in a given line with a lethal phenotype is determined from the insertion points for that line. Often, the precise location of an ORF for a given gene is not known, but predictions are available in genome sections deposited in GenBank. The precise boundaries of that ORF is determined as described in Example 7.

TABLE 4
Insertion Points For Lines With Lethal Phenotypes

| | | | Genome | | Insert | |
|---|---|---|---|---|---|---|
| Gene | Line # | Border | Section | Acc. # | Pt. | Dir. |
| 33 | 62536 | LB | T29H11 | AL049659 | 96460 | D |
| 33 | 62536 | LB | T29H11 | AL049659 | 96470 | U |
| 33 | 94990 | RB | T29H11 | AL049659 | 95809 | U |
| 33 | 94990 | LB | T29H11 | AL049659 | 95739 | D |
| 417 | 75602 | LB | ATCHRIV71 | AL161575 | 139127 | U |
| 417 | 75602 | LB | ATCHRIV71 | AL161575 | 138979 | D |
| 510 | 48882 | RB | MQC12 | AB024036 | 60578 | D |
| 510 | 48882 | LB | MQC12 | AB024036 | 60632 | U |
| 671 | 98507 | LB | T20K9 | AC004786 | 35184 | D |
| 671 | 98507 | LB | T20K9 | AC004786 | 35209 | U |
| 930 | 11129 | AB | T1E22 | AL162874 | 7988 | D |
| 930 | 11129 | LB | T1E22 | AL162874 | 8015 | U |
| 931 | 11206 | LB | YUP8H12R | AC002986 | 12740 | U |
| 931 | 11206 | LB | YUP8H12R | AC002986 | 12716 | D |
| 955 | 11833 | LB | MDC12 | AB008265 | 1978 | D |
| 955 | 11833 | RB | MDC12 | AB008265 | 2023 | U |
| 955 | 16089 | LB | MDC12 | AB008265 | 2141 | D |
| 955 | 16089 | RB | MDC12 | AB008265 | 2367 | U |
| 955 | 123345 | LB | MDC12 | AB008265 | 2340 | D |
| 955 | 123563 | LB | MDC12 | AB008265 | 1936 | U |
| 955 | 123563 | LB | MDC12 | AB008265 | 1924 | D |
| 962 | 16696 | LB | F7A10 | AC027034 | 15603 | D |
| 962 | 16696 | LB | F7A10 | AC027034 | 15663 | U |
| 1019 | 35345 | LB | ATCHRIV30 | AL161518 | 60369 | D |
| 1019 | 35345 | LB | ATCHRIV30 | AL161518 | 60398 | U |
| 1019 | 41510 | LB | ATCHRIV30 | AL161518 | 57633 | U |
| 1019 | 41510 | LB | ATCHRIV30 | AL161518 | 57585 | D |
| 1159 | 21281 | AB | K9I9 | AB013390 | 32147 | D |
| 1159 | 21281 | LB | K9I9 | AB013390 | 32180 | U |
| 1380 | 70615 | AB | T8K14 | AC007202 | 36203 | U |
| 1380 | 70615 | LB | T8K14 | AC007202 | 34862 | D |
| 1413 | 81281 | RB | ATCHRIV12 | AL161500 | 52649 | U |
| 1413 | 81281 | RB | ATCHRIV12 | AL161500 | 52462 | D |
| 1425 | 57819 | LB | ATCHRIV53 | AL161553 | 27669 | D |
| 1425 | 57819 | LB | ATCHRIV53 | AL161553 | 27693 | U |
| 1425 | 96886 | LB | ATCHRIV53 | AL161553 | 27705 | U |
| 1425 | 96886 | RB | ATCHRIV53 | AL161553 | 27695 | D |
| 1456 | 11627 | RB | T24P22 | AC084242 | 7580 | U |
| 1456 | 11627 | RB | T24P22 | AC084242 | 7539 | D |
| 1457 | 62024 | LB | T8F5 | AC004512 | 62716 | U |
| 1457 | 62024 | LB | T8F5 | AC004512 | 62647 | D |
| 3209 | 83826 | LB | MDC11 | AB024034 | 46358 | D |
| 3537 | 40773 | RB | T13O15 | AC010870 | 45038 | D |
| 3537 | 40773 | LB | T13O15 | AC010870 | 45041 | U |
| 7726 | 127024 | RB | K24M9 | AP001303 | 26220 | U |
| 7726 | 127024 | RB | K24M9 | AP001303 | 26175 | D |
| 11197 | 104603 | LB | T20P8 | AC005623 | 19718 | D |
| 11197 | 104603 | LB | T20P8 | AC005623 | 19869 | U |
| 12258 | 118669 | LB | T14G11 | AC002341 | 39667 | U |
| 12258 | 118669 | RB | T14G11 | AC002341 | 39667 | D |
| 19814 | 105512 | LB | MJE7 | AB020745 | 39263 | D |
| 19814 | 105512 | LB | MJE7 | AB020745 | 39265 | U |
| 21858 | 131461 | LB | ATCHRIV80 | AL161584 | 124842 | U |
| 21858 | 131461 | LB | ATCHRIV80 | AL161584 | 124806 | D |
| 25358 | 113413 | LB | K12B20 | AB018107 | 31416 | U |
| 25358 | 113413 | LB | K12B20 | AB018107 | 31389 | D |
| 25358 | 119013 | LB | K12B20 | AB018107 | 29254 | U |
| 25358 | 119013 | LB | K12B20 | AB018107 | 29124 | D |
| 28011 | 23518 | LB | MNB8 | AB018116 | 45722 | U |
| 28011 | 23518 | LB | MNB8 | AB018116 | 45703 | D |

### Example 8

### Identification of cDNAs for Essential Genes

A cDNA for a gene identified as essential is identified using a variety of approaches. This information enables the ORF for a given gene to be identified and used for other experiments including expression of the corresponding protein in heterologous systems.

If there is a full-length cDNA deposited in GenBank or published elsewhere, that sequence may be checked independently using methods described below. Alternatively, the sequence may be considered to be correct.

In some cases, there are published EST sequences that can be assembled to cover the entire ORF from start codon to stop codon. This sequence may be checked independently using methods described below or it may be considered to be correct.

Often part of the cDNA is published and this information can be used to identify the entire ORF. If the 5′ end containing the start codon is known, 3′ RACE is performed to identify the remainder of the cDNA. If the 3′ end containing the stop codon is known, 5′ RACE is performed to identify the remainder of the cDNA. If both the 5′ and the 3′ ends are known, but the sequence between the two ends of the cDNA is not known, PCR is performed with primers hybridizing to each end of the cDNA. In all three of these cases, PCR is performed using template DNA from a GeneRacer (Invitrogen) or a Marathon (Clontech) cDNA library prepared from RNA isolated from seedling tissue. A resulting PCR product is TA-cloned (Original TA-Cloning kit, Invitrogen) and sequenced.

If no part of the cDNA is published, the cDNA is identified by starting from gene model predictions in the annotation for genomic clones or elsewhere. To identify the ORF, primers are designed to the 5′ and 3′ ends of the predicted ORF. PCR is performed using template DNA from a cDNA library prepared from seedling tissue or the pFL61 Arabidopsis cDNA library (Minet et al. (1992) Plant J. 2: 417-422). The resulting PCR product is TA-cloned (Original TA-Cloning kit, Invitrogen) and sequenced. Alternatively, 5′ and 3′ RACE are performed with primers predicted by gene models to be in exons. PCR is performed using template DNA from a GeneRacer (Invitrogen) or a Marathon (Clontech) cDNA library prepared from RNA isolated from seedling tissue. A resulting PCR product is TA-cloned (Original TA-Cloning kit, Invitrogen) and sequenced.

If the cDNA sequence is the same as the sequence predicted in the GenBank annotation, the experiments confirm for the first time the actual ORF. If the cDNA sequence is not the same as the sequence predicted in the GenBank annotation, the experiments identify for the first time the actual ORF. In some cases, more than one cDNA sequence is found for a given gene and both sequences are included in this application.

### Example 9

### Description of Essential Genes

The putative function of the protein encoded by each essential gene is determined from analysis of the ORF in each cDNA. Information from the relevant Arabidopsis genomic section deposited in GenBank is used as a starting point to explore the function of a given gene. This analysis also includes BLAST searches (Altschul et al. (1990) J. Mol. Biol. 215:403-410; Altschul et al. (1997) Nucleic Acids Res. 25:3389-3402) of sequence databases to identify similar proteins. Table 5 describes the putative functions for the essential genes discovered in this application.

TABLE 5
Putative Functions For Essential Genes

| GENE | SEQ ID | | |
|---|---|---|---|
| ID | NO | Putative Function & Similar Genes | References |
| 33 | 1-2 | unknown function, similar genes | Bycroft, M. et al. (1997) Cell 88: 235-242 |
| | | identified in rice (BAB89963.1), | |
| | | tomato, potato, medicago, sorghum, | |
| | | & barley ESTs; may contain S1 | |
| | | RNA binding domain (PFAM | |
| | | 00575) | |
| 417 | 3-4 | unknown function, contains 2 | Ach, R. A. et al. (1997) Plant Cell 9: 1595-1606 |
| | | WD40, G-beta repeat domains | |
| | | (PFAM 00400) | |
| 510 | 5-6 | similar to glucan (1,4-alpha-), | Preiss, J. & Sivak, M. N. (1998) Genet Eng |
| | | branching enzymes (glycogen or | 20: 177-223; Blauth, S. L. et al. (2001) |
| | | starch branching enzymes) | Plant Physiol 125: 1396-1405; Thon, V. J. |
| | | | et al. (1992) J Biol Chem 267: 15224-15228 |
| 671 | 7-8 | may contain a MMR_HSR1, | Vernet, C. et al. (1994) Mamm Genome |
| | | GTPase of unknown function | 5: 100-105 |
| | | domain (PFAM 01926), similar to | |
| | | B. subtilis & E. coli engB | |
| 930 | 9-10 | similar to E. coli 3 prime −5 prime | Marujo, P. E. et al. (2000) RNA 6: 1185-1193; |
| | | exoribonuclease, RNase R (aka | Mohanty, B. K. & Kushner, S R. |
| | | vacB) & other members of RNase II | (2000) Mol Microbiol 36: 982-994; Mian, I. S. |
| | | family | (1997) Nucleic Acids Res. 25: 3187-3195 |
| 931 | 11-12 | similar to D. melanogaster | Majumdar, A. et al. (1997) Genes Dev |
| | | strawberry notch (sno) & human | 11: 1341-1353; Aasland, R. et al. (1995) |
| | | MOP-3; contains a PHD-type zinc | Trends Biochem Sci 20: 56-59 |
| | | finger | |
| 955 | 13-14 | unknown function, similar to Vigna | Kaga, A. & Ishimoto, M. (1998) Mol Gen |
| | | radiata Bng110 (BAB82451) | Genet 258: 378-384 |
| 962 | 15-16 | putative n-calpain-1 large subunit; | Kidd, V. J. et al. (2000) Semin Cell Dev |
| | | similar to maize DEK1 & mouse | Biol. 11: 191-201; Donkor, I. O. (2000) |
| | | calpain 3; calcium activated neutral | Curr Med Chem. 7: 1171-1188; Wang, K. K. |
| | | protease | (2000) Trends Neurosci. 23: 20-26; |
| | | | Lid, S. E. et al. (2002) Proc Natl Acad Sci |
| | | | USA. 99: 5460-5465 |
| 1019 | 17-18 | similar to human AdoMet-binding | Bokar, J. A. et al. (1997) RNA 3: 1233-1247; |
| | | subunit of (N6-adenosine)- | Shah, J. C. & Clancy M. J. (1992) |
| | | methyltransferase (AAG13956) & | Mol Cell Biol 12: 1078-1086; Finnegan, E. J. |
| | | S. cerevisiae IME4 (aka SPO8) | & Kovac, K. A. (2000) Plant Mol Biol. |
| | | | 43: 189-201 |
| 1159 | 19-20 | unknown function, similar to | Bentolila, S. et al. (2002) Proc Natl Acad |
| | | petunia Rf-PPR592 fertility restorer | Sci USA 99: 10887-10892; Small, I. D. & |
| | | protein, contains PPR domains | Peeters, N. (2000) Trends Biochem Sci |
| | | (PFAM 01535), member of large | 25: 46-47; Fisk, D. G. et al. (1999) EMBO J |
| | | gene family in Arabidopsis | 18: 2621-2630; Coffin, J. W. (1997) Curr. |
| | | | Genet. 32: 273-280 |
| 1380 | 21-22 | unknown function, similar to | Bentolila, S. et al. (2002) Proc Natl Acad |
| | | petunia Rf-PPR592 fertility restorer | Sci USA 99: 10887-10892; Small, I. D. & |
| | | protein, contains a SMR (Small | Peeters, N. (2000) Trends Biochem Sci |
| | | MutS-related) domain and PPR | 25: 46-47; Fisk, D. G. et al. (1999) EMBO J |
| | | repeats (PFAM 01535), member of | 18: 2621-2630; Coffin, J. W. (1997) Curr. |
| | | large gene family in Arabidopsis | Genet. 32: 273-280; Moreira, D. & |
| | | | Philippe, H. (1999) Trends Biochem Sci |
| | | | 24: 298-300 |
| 1413 | 23-24 | putative leucyl tRNA synthetase | Thompson, L. H. et al. (1973) |
| | | | Proc Natl Acad Sci USA 70: 3094-3098; |
| | | | Hartlein, M. & Madern, D. (1987) Nucleic |
| | | | Acids Res 15: 10199-10210; Labouesse, M. |
| | | | (1990) Mol Gen Genet 224: 209-221 |
| 1425 | 25-26 | similar to RNA splicing factors | Gray, T. A. et al. (1999) Nucleic Acids Res |
| | | small nuclear ribonucleoprotein B, | 27: 4577-4584; Ozcelik, T. et al. (1992) |
| | | B′, & N in humans and other | Nat. Genet. 2: 265-269 |
| | | animals | |
| 1456 | 27-28 | putative chorismate synthase | Schaller, A. et al. (1991) J Biol Chem. |
| | | | 266: 21434-21438; Gorlach, J. et al. (1993) |
| | | | Plant Mol Biol. 23: 707-716; Gorlach, J. et |
| | | | al. (1995) Plant J. 8: 451-456; Braun, M. et |
| | | | al. (1996) Planta 200: 64-70 |
| 1457 | 29-30 | putative ABC transporter | Stacey, G. et al. (2002) Trends Plant Sci. |
| | | | 7: 257-263; Smith, P. et al. (2002) Mol Cell |
| | | | 10: 139-149; Fath, M. J. & Kolter, R. |
| | | | (1993) Microbiol Rev. 57: 995-1017 |
| 3209 | 31-32 | unknown function, may contain a | Ach, R. A. et al. (1997) Plant Cell 9: 1595-1606; |
| | | WD40, G-beta repeat domain | Garcia-Lozano, J. R. et al. (1997) |
| | | (PFAM 00400), weak similarity to | Clin Exp Immunol 107: 501-506 |
| | | human autoantigen (RCD-8) | |
| 3537 | 33-34 | similar to maize CRS1, required for | Till, B. et al. (2001) RNA 7: 1227-1238; |
| | | splicing atpF group II intron in | Vogel, J., Borner, T. et al. (1999) Nucleic |
| | | chloroplasts, which has a seedling | Acids Res. 27: 3866-3874 |
| | | lethal phenotype | |
| 7726 | 35-36 | unknown function, contains single- | Meyer, R. R. & Laine, P. S. (1990) |
| | | stranded binding protein domain | Microbiol Rev 54: 342-380; Meyer, R. R. et |
| | | (PFAM 00436), weak similarity to | al. (1979) Proc Natl Acad Sci USA. |
| | | E. coli SSB2 | 76: 1702-1705; Ruvolo, P. P. et al. (1991) |
| | | | Proteins 9: 120-134 |
| 11197 | 37-38 | Arabidopsis COP9 complex subunit | Fu, H. et al. (2001) EMBO J. 20: 7096-7107; |
| | | CSN2, contains a PCI/PINT domain | Bech-Otschir, D. et al. (2002) J. Cell |
| | | (PFAM 01399) | Sci. 115: 467-473; Chamovitz, D. & |
| | | | Glickman, M. (2002) Curr Biol. 12: R232 |
| 12258 | 39-40 | unknown function, contains a | none |
| | | DUF231 domain (PFAM 03005), | |
| | | member of large gene family in | |
| | | Arabidopsis | |
| 19814 | 41-42 | unknown function | none |
| 21858 | 43-44 | unknown function, contains PPR | Small, I. D. & Peeters, N. (2000) Trends |
| | | domains (PFAM 01535), member of | Biochem Sci 25: 46-47; Fisk, D. G. et al. |
| | | large gene family in Arabidopsis | (1999) EMBO J 18: 2621-2630; Coffin, J. W. |
| | | | (1997) Curr. Genet. 32: 273-280 |
| 25358 | 45-46 | similar to Xenopus chromosome | Cabello, O. A. et al. (2001) Mol Biol Cell |
| | | condensation protein XCAP-G, 130 kD | 12: 3527-3537; Kimura, K. et al. (2001) J |
| | | subunit of the 13S condensin | Biol Chem 276: 5417-5420 |
| | | complex, & human hCAP-G | |
| 28011 | 47-48 | unknown function, contains | Lamb, J. R. et al. (1995) Trends Biochem |
| | | tetratricopeptide repeat (TPR) | Sci 20: 257-259; Das, A. K. et al. (1998) |
| | | domains (PFAM 00515) | EMBO J 17: 1192-1199; Goeb, l M. & |
| | | | Yanagida, M. (1991) Trends Biochem Sci |
| | | | 16: 173-177 |

### Example 10

### Expression of Recombinant Essential Proteins in E. coli

The coding region of each of the essential proteins, corresponding to cDNA clones of odd-numbered SEQ ID NO: 1-96, is subcloned into an appropriate expression vector, and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of the essential protein is confirmed. Recombinant protein is isolated using standard techniques.

### Example 11

### In Vitro Binding Assays

Recombinant protein for each of the essential genes described in this application is obtained, for example, according to Example 10. The protein is immobilized on chips appropriate for ligand binding assays using techniques that are well known in the art. The protein immobilized on the chip is exposed to sample compound in solution according to methods well know in the art. While the sample compound is in contact with the immobilized protein, measurements capable of detecting protein-ligand interactions are conducted. Examples of such measurements are SELDI, biacore and FCS, described above. Compounds found to bind the protein are readily discovered in this fashion and are subjected to further characterization.

The above-disclosed embodiments are illustrative. This disclosure of the invention will place one skilled in the art in possession of many variations of the invention. All such obvious and foreseeable variations are intended to be encompassed by the present invention.

What is claimed is:

1. A method of identifying a herbicidal compound, comprising:
a) combining a polypeptide comprising an amino acid sequence at least 90% identical to an amino acid sequence selected from the group consisting of the even numbered SEQ ID NOs:2-48 with a compound to be tested for the ability to bind to said polypeptide, under conditions conducive to binding;
b) selecting a compound identified in (a) that binds to said polypeptide;
c) applying a compound selected in (b) to a plant to test for herbicidal activity; and
d) selecting a compound identified in (c) that has herbicidal activity.

2. The method according to claim 1, wherein said polypeptide comprises an amino acid sequence at least 95% identical to an amino acid sequence selected from the group consisting of the even numbered SEQ ID NOs:2-48.

3. The method according to claim 2, wherein said polypeptide comprises an amino acid sequence at least 99% identical to an amino acid sequence selected from the group consisting of the even numbered SEQ ID NOs:2-48.

4. The method according to claim 3, wherein said polypeptide comprises an amino acid sequence selected from the group consisting of the even numbered SEQ ID NOs:2-48.

5. A method of identifying a herbicidal compound, comprising:
c) combining a polypeptide comprising an amino acid sequence at least 90% identical to an amino acid sequence selected from the group consisting of the even numbered SEQ ID NOs:2-48 with a compound to be tested for the ability to inhibit the activity of said polypeptide, under conditions conducive to inhibition;
d) selecting a compound identified in (a) that inhibits the activity of said polypeptide;
c) applying a compound selected in (b) to a plant to test for herbicidal activity; and
d) selecting a compound identified in (c) that has herbicidal activity.

6. The method according to claim 5, wherein said polypeptide comprises an amino acid sequence at least 95% identical to an amino acid sequence selected from the group consisting of the even numbered SEQ ID NOs:2-48.

7. The method according to claim 6, wherein said polypeptide comprises an amino acid sequence at least 99% identical to an amino acid sequence selected from the group consisting of the even numbered SEQ ID NOs:2-48.

8. The method according to claim 7, wherein said polypeptide comprises an amino acid sequence selected from the group consisting of the even numbered SEQ ID NOs:2-48.

9. A method for killing or inhibiting the growth or viability of a plant, comprising applying to the plant a herbicidal compound identified according to the method of claim 1.

10. A method for killing or inhibiting the growth or viability of a plant, comprising applying to the plant a herbicidal compound identified according to the method of claim 5.
